(19) United States
(12) Patent Application Publication
(10) Pub. No.: US 2006/0188874 A1
(43) Pub. Date: Aug. 24, 2006

(54) Methods and compositions for colorimetrically assessing peptide characteristics

(76) Inventors: Ileana Isabel Nuiry, Boulder, CO (US); Roger P. Micheli, Louisville, CO (US)

Correspondence Address: Roche Colorado Corporation, 2075 North 55th Street, Boulder, CO, 80301, US

(21) Appl. No.: 11/317,871

(22) Filed: Dec. 23, 2005

Related U.S. Application Data
(60) Provisional application No. 60/640,717, filed on Dec. 30, 2004.

(51) Int. Cl. C12Q 1/70; G01N 33/00

(52) U.S. Cl. 435/5; 436/86

(57) ABSTRACT

Methodologies and compositions useful for calorimetrically assessing peptide characteristics. By way of example, the methodologies can be used to qualitatively and quantitatively assess the degree to which a peptide sample incorporates beta sheet content. The colorimetric information also information is useful to predict solubility, formulating, and other peptide characteristics impacted by beta sheet aggregation. The methodologies can also be practiced for quality control during peptide manufacture. The methodologies can be practiced to determine whether it might be desirable to subject a peptide sample to a deaggregation treatment.

### PRIORITY CLAIM

The present non-provisional patent Application claims priority under 35 USC §119(e) from U.S. Provisional Patent Application having Ser. No. 60/640,717, filed on Dec. 30, 2004, by Nuiry and titled METHODS AND COMPOSITIONS FOR COLORIMETRICALLY ASSESSING PEPTIDE CHARACTERISTICS, wherein the entirety of said provisional patent application is incorporated herein by reference.

### FIELD OF THE INVENTION

The present invention relates to methodologies and compositions useful for calorimetrically assessing peptide characteristics. By way of example, the methodologies can be used to qualitatively and quantitatively assess the degree to which a peptide sample incorporates beta sheet structure. The information is useful to predict solubility, formulating, and other peptide characteristics impacted by beta sheet structure and aggregation associated with such structure. The methodologies can also be practiced for quality control during peptide manufacture.

### BACKGROUND OF THE INVENTION

Proteins and other peptides are not only useful in the treatment a wide variety of diseases but also are useful research tools for the discovery of pharmaceutically active compounds. Many proteins and peptides of interest are readily soluble in aqueous solution at physiological pH. For example, the FUZEON®& peptide (also known as enfuvirtide or T-20), which is a synthetic, 36-amino-acid peptide, the hybrid peptide T-1249, and derivatives and counterparts of these peptides, have proven beneficial as fusion inhibitors in the treatment of the human immunodeficiency virus (HIV) and the acquired immune deficiency syndrome (AIDS). The FUZEON® peptide and its derivatives are the first inhibitors of HIV to demonstrate consistent, potent activity in persons infected with HIV. Kilby et al. (1998) Nat Med 4:1302 and Kilby et al. (2002) AIDS Res Hum Retroviruses 18:685.

Fusion inhibitors such as the T-20 and T-1249 peptides bind to a region of the glycoprotein 41 envelope of HIV type 1 (HIV-1) that is involved in the fusion of the virus with the membrane of the CD4+ host cell. Wild et al. (1993) AIDS Res. Hum. Retroviruses 9:1051. Fusion inhibitors remain outside the cell and block HIV-1 prior to HIV-1 entering the cell. The FUZEON® peptide and its derivatives minimize drug interactions, side effects and cytotoxicity by potently and selectively inhibiting HIV-1 in vitro.

Another example of a peptide of interest is the Glucagon-Like Peptide 1 (GLP-1). GLP-1 peptide is a 37 amino acid peptide that is secreted by the L-cells of the intestine in response to food ingestion. It has been found to stimulate insulin secretion (insulinotropic action), thereby causing glucose uptake by cells and decreased serum glucose levels (see, e.g., Mojsov, S., (1992) Int. J. Peptide Protein Research, 40:333-343). However, GLP-1 is poorly active. A subsequent endogenous cleavage between the 6th and 7th position produces a more potent biologically active GLP-1(737)OH peptide. Numerous other GLP-1 counterparts are also known. Because of their ability to stimulate insulin secretion, GLP compounds show great promise as agents for the treatment of diabetes, obesity, and related conditions. See also PCT Patent Publication WO 01/55213, hereby incorporated in its entirety by reference.

Proteins and peptides are inherently unstable molecules due to their multiple functional groups, which complicate recombinant reproduction and purification of homogeneous protein preparations having the pharmacologically desired biological and physiochemical characteristics. Senderoff et al. (1997) J. Pharm. Sci. 87:183. The therapeutic potential of peptides and proteins is dependent on the production and purification of their active forms in commercially viable quantities.

A key characteristic impacting the manufacturability and/or use of peptide products concerns the solubility of these products in aqueous media at physiological pH. As used herein, the term “physiological pH” refers to a pH in the range of from about 6.5 to about 7.5, preferably about 6.9 to about 7.5, more preferably about 7.1 to about 7.4. Such solubility is highly desirable for a variety of reasons. Firstly, most environments of intended use within the body of human or animal patients constitute aqueous media at physiological pH. Secondly, most peptides tend to be more biologically active when soluble in such media.

Biologically active peptide compounds with identical sequences of amino acids (i.e., identical primary structure) nonetheless can exist coiled in at least two different forms (i.e., secondary structures), namely beta sheets and/or alpha helices. A peptide sample often may incorporate both structures to some degree. The solubility characteristics of a peptide may significantly depend to a large degree upon the relative amounts of these structures that are present in a peptide sample. With increasing beta sheet structure, the molecular weight of a peptide appears to be multiples of the theoretical peptide weight, and such peptides tend to be soluble in aqueous media only under strongly acidic or strongly basic conditions. Peptides with increasing amounts of beta sheet structure also tend to have more tertiary structure. Additionally, the β-sheet structure tends to be much less biologically active than the a-helical structure. As another drawback, peptides with greater amounts of beta sheet structure may tend to be more difficult to handle, filter, compound, or otherwise process.

The terms “aggregation” and “deaggregation” (deaggregation is sometimes referred to as “disaggregation.”) are used to describe peptides and their structures. Generally, a peptide including undue amounts of beta sheet structure is referred to as being aggregated, whereas a peptide including substantial amounts of alpha helical structure is referred to as being deaggregated. A peptide can be more or less aggregated or deaggregated from another peptide or other version of the same peptide depending upon the relative amounts of beta sheet and alpha helical structures that are present in the samples being compared.

It should be noted that characterizing a peptide as having one structure and/or the other, or as being more or less aggregated or deaggregated, does not refer to the purity of the peptide. Rather, the two forms are different from each other analogously to the way that polymorphs are different from each other.

It tends to be most desirable to produce peptides that are relatively more deaggregated due to solubility and biological activity concerns. To that end, beta-sheet to alpha-helix coil transition specifically related to peptides has been studied in the art. See, e.g., Kim et al. (1994) J. of Pharm. Sci. 83:1175 and Senderoff et al. (1998) J. Pharm. Sci. 87:183. Unfortunately, some peptides, such as the T-1249 and GLP-1 peptides and their counterparts, tend to be susceptible to the formation of beta sheet, tertiary structures, and associated aggregation. Such peptides are especially sensitive to aggregation (e.g., such as due to formation of undue amounts of beta sheet structure) during precipitation. This frustrates the realization of the pharmaceutical potential of peptide compounds, because this potential is dependent on the production of the active form of these compounds in commercially viable quantities without contamination with significant quantities of byproducts of the inactive form.

Accordingly, it is desired to be able to manufacture a peptide in such way that allows the relative degree to which the resultant peptide is deaggregated or aggregated.

Whether precipitated with or without significant beta sheet structure, precipitated peptide can be difficult to filter. In some instances, the precipitate is too gel-like so that the incipient filter cake holds onto water too tenaciously, making filtering and washing of impurities impractical. It would be highly desirable to avoid this gel-like state and/or convert the gel-like peptide into a more filterable form. With respect to deaggregated peptide, it would be desirable to accomplish this without causing undue aggregation of the precipitated peptide.

Because of the problems associated with a peptide that incorporates beta sheet content, it also would be highly desirable to have a cost effective, accurate, robust methodology that would allow peptide material to be qualitatively and quantitatively tested for beta sheet content.

### SUMMARY OF THE INVENTION

The present invention relates to methodologies and compositions useful for calorimetrically assessing peptide characteristics. By way of example, the methodologies can be used to qualitatively and quantitatively assess the degree to which a peptide sample incorporates beta sheet content. The colorimetric information also is useful to predict solubility, formulating, and other peptide characteristics impacted by beta sheet aggregation. The methodologies can also be practiced for quality control during peptide manufacture. The methodologies can be practiced to determine whether it might be desirable to subject a peptide sample to a deaggregation treatment.

In one aspect, the present invention relates to a method of processing a peptide. A portion of the peptide is combined with a calorimetric reagent comprising a dye that colorimetrically and selectively interacts more strongly with a first structure of the peptide relative to a second structure of the peptide. The first structure is substantially insoluble in aqueous media at physiological pH, and the second structure is substantially soluble in aqueous media at physiological pH. Colorimetric information is then obtained that is indicative of whether the peptide sample contains an undesired amount of the first structure. The information is used to determine whether to subject the peptide to a deaggregation treatment.

In another aspect, the present invention relates to a method of processing a peptide. A portion of the peptide is combined with a colorimetric reagent comprising a dye that colorimetrically and selectively interacts with the peptide in a manner that qualitatively correlates to an amount of beta sheet structure of the peptide. Colorimetric information is obtained that is indicative of whether the peptide sample contains an undesired amount of the structure. The information is used to determine whether to subject the peptide to a deaggregation treatment.

In another aspect, the present invention relates to a method of processing a peptide. A portion of the peptide is combined with a calorimetric reagent comprising a dye that calorimetrically and selectively interacts with the peptide in a manner that quantitatively correlates to an amount of beta sheet structure of the peptide. Colorimetric information is obtained that is indicative of whether the peptide sample contains an undesired amount of the structure. The information is used to determine whether to subject the peptide to a deaggregation treatment.

In another aspect, the present invention relates to a method of calorimetrically testing a peptide. The peptide is combined with a colorimetric reagent that selectively interacts with a first structure of a peptide relative to a second structure of the peptide. Colorimetric information is obtained that is indicative of the formulation characteristics of the peptide. The colorimetric information is used to assess the formulation characteristics of the peptide.

In another aspect, the present invention relates to a method of monitoring the quality of peptide synthesis. The peptide is combined with a colorimetric reagent that selectively interacts with a first structure of a peptide relative to a second structure of the peptide. Colorimetric information is then used to determine information indicative of whether the peptide satisfies a quality control specification.

In another aspect, the present invention relates to a method of monitoring the quality of peptide preparation. A sample mixture is prepared according to steps comprising combining ingredients comprising a peptide and a colorimetric reagent. Spectroscopic information for the sample mixture is obtained. The spectroscopic information for the mixture is compared to spectroscopic information for a reference mixture. The comparison information is used to assess a quality characteristic of the peptide.

In another aspect, the present invention relates to a method of creating a quantitative, spectroscopic specification for a peptide. A plurality of peptide samples are provided having differing degrees of solubility in aqueous media at physiological pH. Each peptide sample is respectively combined with a colorimetric reagent to provide respective peptide/colorimetric reagent mixtures. Information indicative of the respective spectroscopic characteristics of the mixtures is obtained. The information is used to provide the quantitative, spectroscopic, peptide specification.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The methodologies of the present invention would be very suitable with respect to a wide range of peptides and proteins. A preferred class of peptides of the present invention are those that incorporate from about 2 to about 100, preferably from about 4 to about 50, residues of one or more amino acids. Residues of one or more other monomeric, oligomeric, and/or polymeric constituents optionally may also be incorporated into a peptide. Non-peptide bonds also may be present. For instance, the peptides of the invention may be synthesized to incorporate one or more non-peptide bonds. These non-peptide bonds may be between amino acid residues, between an amino acid and a non-amino acid residue, or between two non-amino acid residues. These alternative non-peptide bonds may be formed by utilizing reactions well known to those in the art, and may include, but are not limited to imino, ester, hydrazide, semicarbazide, and azo bonds, to name but a few.

The amino acids from which the peptides are derived may be natural or non-natural. The twenty, common, naturally-occurring amino acids residues and their respective one-letter symbols are as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine). Naturally-occurring, rare amino acids are also contemplated and include, for example, selenocysteine, pyrrolysine.

Non-natural amino acids includes organic compounds having a similar structure and reactivity to that of a naturally-occurring amino acid include, for example, D-amino acids, beta amino acids, gamma amino acids; cyclic amino acid analogs, propargylglycine derivatives, 2-amino-4-cyanobutyric acid derivatives, Weinreb amides of α-amino acids, and amino alcohols. Incorporation of such amino acids into a peptide may serve to increase the stability, reactivity and/or solubility of the peptides of the invention.

Representative examples of peptides that may be processed in accordance with the present invention include peptides with fusion inhibiting activity such as enfuvirtide (also known as the T-20 peptide) and the T-1249 peptide; peptides that stimulate insulin secretion such as glucagons-like peptide 1 (GLP-1) and its more potent analog the GLP-1 (7-37)OH peptide; Oxytocin (9 C SP); vasopressin: Felypressin, Pitressin (9 C), Lypressin (9 C), Desmopressin (9 C SP), Terlipressin (12 C); Atosiban (9 C); adrenocorticotropic hormone (ACTH; 24 C); Insulin (51 recombinant or semisynthesis), Glucagon (29 recombinant SP); Secretin (27); calcitonins: human calcitonin(32 C), salmon calcitonin(32 C SP), eel calcitonin(32 C SP), dicarba-eel (elcatonin) (31 C SP); luteinizing hormone-releasing hormone (LH-RH) and analogues: leuprolide(9 C), deslorelin(9 SP), triptorelin(10 SP), goserelin(10 SP), buserelin(9 SP); nafarelin(10 C), cetrorelix(10 SP), ganirelix(10 C), parathyroid hormone (PTH) (34 SP); human coriticotropin-releasing factor(41 SP), ovine coriticotropin-releasing factor(9 C SP); growth hormone releasing factor(9 C SP); somatostatin(9 C SP); lanreotide(9 C SP), octreotide(9 C SP); thyrotropin releasing hormone (TRH) (9 C SP); thymosin α-1(9 C SP); thymopentin (TP-5) (9 C SP); cyclosporin(9 C SP); integrilin(9 C SP); angiotensin-converting enzyme inhibitors: enalapril(9 C SP), lisinopril(9 C SP); fragments of such peptides; counterparts of such peptides, and the like.

Preferably, the principles of the invention are practiced with respect to peptides with fusion inhibiting activity such as enfuvirtide (also known as the T-20 peptide) and the T-1 249 peptide; peptides that stimulate insulin secretion such as glucagons-like peptide 1 (GLP-1) and its more potent analog the GLP-1 (7-37)OH peptide; fragments of such peptides; and counterparts of these. More preferably, the principles of the invention are practiced with respect to the T-1249 peptide, GLP-1 peptide, the GLP-1 (7-37) OH peptide, fragments of such peptides, and counterparts of these inasmuch as these peptide materials are more prone to solubility issues, such as may result via aggregation of the peptide material into insoluble beta sheet structures. Most preferably, the principles of the invention are practiced with respect to the T-1249 peptide, fragments thereof, and counterparts thereof.

As used herein, a “counterpart” of a peptide refers to a compound derived from another peptide or peptide counterpart and comprising a backbone incorporating a sequence of 2 or more amino acid residues. Peptide counterparts include but are not limited to peptide analogs, peptide derivatives, follow on compounds, fusion compounds, and the like. As used herein, a peptide analog generally refers to a peptide having a modified amino acid sequence such as by one or more amino acid substitutions, deletions, inversions, and/or additions relative to another peptide or peptide counterpart. Substitutions preferably may be conservative or highly conservative. A conservative substitution refers to the substitution of an amino acid with another that has generally the same net electronic charge and generally the same size and shape. For instance, amino acids with aliphatic or substituted aliphatic amino acid side chains have approximately the same size when the total number carbon and heteroatoms in their side chains differs by no more than about four. They have approximately the same shape when the number of branches in the their side chains differs by no more than about one or two. Amino acids with phenyl or substituted phenyl groups in their side chains are considered to have about the same size and shape. Listed below are five groups of amino acids. Replacing an amino acid in a compound with another amino acid from the same groups generally results in a conservative substitution.

Group I: glycine, alanine, valine, leucine, isoleucine, serine, threonine, cysteine, methionine and non-naturally occurring amino acids with C1-C4 aliphatic or C1-C4 hydroxyl substituted aliphatic side chains (straight chained or monobranched).

Group II: glutamic acid, aspartic acid and nonnaturally occurring amino acids with carboxylic acid substituted C1-C4 aliphatic side chains (unbranched or one branch point).

Group III: lysine, ornithine, arginine and nonnaturally occurring amino acids with amine or guanidino substituted C1-C4 aliphatic side chains (unbranched or one branch point).

Group IV: glutamine, asparagine and non-naturally occurring amino acids with amide substituted C1-C4 aliphatic side chains (unbranched or one branch point).

Group V: phenylalanine, phenylglycine, tyrosine and tryptophan.

A “highly conservative substitution” is the replacement of an amino acid with another amino acid that has the same functional group in the side chain and nearly the same size and shape. Amino acids with aliphatic or substituted aliphatic amino acid side chains have nearly the same size when the total number carbon and heteroatoms in their side chains differs by no more than two. They have nearly the same shape when they have the same number of branches in the their side chains. Examples of highly conservative substitutions include valine for leucine, threonine for serine, aspartic acid for glutamic acid and phenylglycine for phenylalanine.

A peptide derivative generally refers to a peptide, a peptide analog, or other peptide counterpart having chemical modification of one or more of its side groups, alpha carbon atoms, terminal amino groups, and/or terminal carboxyl acid group. By way of example, a chemical modification includes, but is not limited to, adding chemical moieties, creating new bonds, and/or removing chemical moieties. Modifications at amino acid side groups include, without limitation, acylation of lysine e-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl (e.g., —CO-lower alkyl) modifications. Modifications of the terminal carboxy group include, without limitation, the amide, lower alkyl amide, dialkyl amide, and lower alkyl ester modifications. Thus, partially or wholly protected peptides constitute peptide derivatives.

Enfurvitide, also known as T-20, is a peptide that has the 36 amino acid sequence (reading from amino, NH₂ to carboxy, COOH, terminus)
Acetyl-YTSLIHSLIEESQNQQEKNEQELLELDKWASLWNWF-NH₂ (SEQ ED NO. 1)

Representative peptide fragments of enfuvirtide include, but are not limited to, those having amino acid sequences as depicted in Table 1 below or their counterparts. For example, in the table, the amino acid in the 36^{th} position, which is F, may have a carboxylic acid terminus as in the case of the prime metabolite, or it may be modified as the amide in the case of the T-20 peptide itself.

TABLE 1

| | | Corresponding |
|---|---|---|
| | Seq. ID | numbered amino acid |
| Amino Acid Sequence | No. | sequence of T-20 |
| YTSLIHSL | 2 | 1-8 |
| YTSLIHSLIEESQNQ | 3 | 1-15 |
| YTSLIHSLIEESQNQQ | 4 | 1-16 |
| YTSLIHSLIEESQNQQEK | 5 | 1-18 |
| IEESQNQ | 6 | 9-15 |
| IEESQNQQ | 7 | 9-16 |
| QEKNEQELLELDKWASLWNW | 8 | 16-35 |
| QEKNEQELLELDKWASLWNWF | 9 | 16-36 |
| EKNEQEL | 10 | 17-23 |
| EKNEQELLEL | 11 | 17-26 |
| EKNEQELLELDKWASLWNWF | 12 | 17-36 |
| NEQELLELDKWASLWNW | 13 | 19-35 |
| NEQELLELDKWASLWNWF | 14 | 19-36 |
| LELDKWASLWNW | 15 | 24-35 |
| LELDKWASLWNWF | 16 | 24-36 |
| DKWASLWNW | 17 | 27-35 |
| DKWASLWNWF | 18 | 27-36 |
| EKNEQELLELDKWASLWNW | 19 | 17-35 |

Representative counterparts of enfuvirtide are described in U.S. Pat. Nos. 5,464,933; 5,656,480; 6,281,331; 6,469,136; and 6,015,881; or PCT Publication No. WO 96/19495, all of which are incorporated herein by reference in their respective entireties. The synthesis of peptides having T-20 activity and peptide intermediates used to prepare peptides having T-20 activity also are described in U.S. Pat. Nos. 6,469,136; 6,281,331; 6,015,881; 5,464,933; 5,656,480 and PCT Publication No. WO 96/19495. The T-20 peptide and methods of making the T-20 peptide and fragments thereof are described in Assignee's co-pending, U.S. provisional application filed concurrently herewith titled IMPROVED SYNTHESIS USING PEPTIDE INTERMEDIATE FRAGMENTS in the names of Hiralal N. Khatri et al. and bearing Attorney Docket No. RCC0016/P1, the entirety of which is incorporated herein by reference; and in Assignee's co-pending, U.S. provisional application filed concurrently herewith titled IMPROVED SYNTHESIS USING PEPTIDE INTERMEDIATE FRAGMENTS in the names of Hiralal N. Khatri et al., and bearing Attorney Docket No. RCC0018/P1, the entirety of which is incorporated herein by reference.

By custom in the art, a peptide such as T-20 according to Seq. ID No. 1 or the like may be referred to by a designation such as T-20 (1-36), wherein the amino terminus has been assigned the lower number (here the number 1) and the carboxy terminus is assigned the higher number. As a further example, a peptide fragment according to Seq. ID No.2 may be designated as T-20 (1-8). When not specified, the C-terminal is usually considered to be in the traditional carboxyl form. For purposes of clarity, superscript numerals may be included in a sequence to assist in locating amino acid residues at a particular position. This is shown, for instance, in Seq. ID. No.35, below. In the nomenclature used herein to designate peptide counterparts in which an amino acid substitution, the substituting amino acid and its position is indicated prior to the parent structure. For example, Val⁸-GLP-1(7-37)OH designates a GLP-1 analog in which the alanine normally found at position eight of GLP-1(7-37)OH has been replaced with valine.

The T-1249 peptide has the 39 amino acid sequence (reading from acetyl terminus (corresponding to the amino terminus) to the amide terminus (corresponding to the carboxy terminus)

(SEQ. ID NO. 20)

Acetyl-WQEWEQKITALLEQAQIQQE KNEYELQKLDKWASLWEWF-
NH₂

Representative peptide fragments of T-1249 peptide include, but are not limited to, those having amino acid sequences as depicted in Table 2 below as well as counterparts of these. For example, in the table, the amino acid in the 39^{th} position, which is F, may have a carboxylic acid terminus as in the case of the prime metabolite, or it may be modified as the amide in the case of the T-1249 peptide itself.

TABLE 2

| | | Corresponding |
|---|---|---|
| | | Numbered Amino |
| | | Acid Sequence of T- |
| Amino Acid Sequence | Seq. ID No. | 1249 |
| WQEWEQKITALLEQAQIQQE | 21 | 1-20 |
| KNEYELQKLDKWASLWEW | 22 | 21-38 |
| KNEYELQKLDKWASLWEWF | 23 | 21-39 |
| IQQEKNEYELQKLDKWASL | 24 | 17-35 |
| WQEWEQKITALLEQAQI | 25 | 1-17 |
| WQEWEQKITALLEQAQ | 26 | 1-16 |
| QQEKNEYELQKLDKWASLW | 27 | 18-36 |
| QQEKNEYELQKLDKWASL | 28 | 18-35 |
| IQQEKNEYELQKLDKWASLW | 29 | 17-36 |
| WQEWEQKITALL | 30 | 1-12 |
| EQAQIQQE KNEYEL | 31 | 13-26 |
| QKLDKWASLWEW | 32 | 27-38 |
| QKLDKWASLWEWF | 33 | 27-39 |
| EQAQIQQE | 34 | 13-39 |
| KNEYELQKLDKWASLWEWF | | |

The T-1249 peptide and methods of making the T-1249 peptide and fragments thereof are described in U.S. Pat. No. 6,469,136, incorporated herein by reference in its entirety. The T-1249 peptide and methods of making the T-1249 peptide and fragments thereof also are described in Assignee's co-pending, U.S. provisional application filed concurrently herewith titled IMPROVED SYNTHESIS USING PEPTIDE INTERMEDIATE FRAGMENTS in the names of Hiralal N. Khatri et al., and bearing Attorney Docket No. RCC0019/P1, the entirety of which is incorporated herein by reference.

Generally, the GLP-1 peptide and its counterparts are peptides that have from about twenty-five to about thirty-seven amino acid residues and that stimulate insulin secretion upon food ingestion. Naturally occurring Glucagon-Like Peptide 1 (GLP-1) itself is a 37 amino acid peptide that is secreted by the L-cells of the intestine in response to food ingestion. It has been found to stimulate insulin secretion (insulinotropic action), thereby causing glucose uptake by cells and decreased serum glucose levels (see, e g., Mojsov, S., (1992) Int. J. Peptide Protein Research, 40:333-343).

However, GLP-l(1-37) itself is poorly active. A subsequent endogenous cleavage between the 6th and 7th position produces the more potent, naturally occurring, biologically active GLP-1(7-37)OH peptide, which has the following amino acid sequence:

(SEQ ID NO.35)

⁷His- Ala-Glu-¹⁰Gly-Thr- Phe-Thr- Ser-¹⁵Asp-Val-
Ser-Ser-Tyr-²⁰Leu-Glu-Gly-Gln-Ala-²⁵Ala-Lys-Glu-
Phe-Ile-³⁰Ala-Trp-Leu-Val-Lys-³⁵Gly-Arg-³⁷Gly-COOH

Many other GLP-1 counterparts also are known. For example, GLP-1(7-36)NH₂ is the amide form of GLP-1(7-36). Val⁸-GLP-1 (7-37)OH is a synthetic GLP-1 (7-37)OH analog in which alanine at position 8 has been replaced with valine. Thr¹⁶-Lys¹⁸-GLP-1(7-37)OH is a synthetic GLP-1(7-37)OH analog in which valine at position sixteen and serine at position eighteen have been replaced with threonine and lysine, respectively. Others include, but are not limited to: GLP-1(7-34), GLP-1(7-35), GLP-1(7-36)NH₂, Gln⁹-GLP-1(7-37), d-Gln⁹-GLP-1(7-37), Lys¹⁸-GLP-1(7-37), Gly⁸-GLP-1(7-36)NH₂ Gly⁸-GLP-1(7-37)OH, Val⁸-GLP-1(7-36)NH₂. Val⁸-GLP-1(7-37)OH, Met⁸-GLP-1(7-36)NH₂, Met⁸-GLP-1(7-37)OH, Ile⁸-GLP-1(7-36)NH₂, Ile⁸-GLP-1(7-37)OH, Thr⁸-GLP-1(7-36)NH₂, Thr⁸-GLP1(7-37)OH, Ser8-GLP-1(7-36)NH₂. Ser⁸-GLP-1(7-37)OH, Asp⁸-GLP-1(7-36)NH₂. Asp⁸-GLP-1(7-37)OH, Cys⁸-GLP-1(7-36)NH₂, Cys⁸-GLP-1(7-37)OH, Thr⁹-GLP-1(7-37), D-Thr⁹-GLP-1(7-37), Asn⁹-GLP-1(7-37), D-Asn⁹-GLP-1(7-37), Ser²²-Arg²³-Arg²⁴-Gln²⁶-GLP-1(7-37), Arg²³-GLP-1(7-37), Arg²⁴-GLP-1(7-37), Gly⁸-Gln²¹-GLP-1(7-37)OH, and the like.

The GLP-1 peptide and its counterparts have also been described in U.S. Pat. No. 5,705,483; U.S. Pat. No. 5,512,549; U.S. Pat. No. 5,188,666; WO 91/11457; WO 98/08871; and WO 01/55213; each of which is incorporated herein by reference in its entirety. Because of their ability to stimulate insulin secretion, GLP compounds show great promise as agents for the treatment of diabetes, obesity, and related conditions.

Significantly, the methodologies of the present invention may be practiced to convert the more insoluble form of a peptide to a more soluble form. The methodology is robust, consistent, and facilitates the large scale, commercial production of peptides. Without wishing to be bound, a rationale may be suggested to explain the conversion of the insoluble peptide form to one that is soluble. It has been reported that the insoluble form of GLP-1 compounds is characterized by the presence of relatively greater amounts of intramolecular and intermolecular beta sheet structure, which results in peptide aggregation and insolubility. In contrast, the more soluble form is characterized by the presence of relatively greater amounts of alpha helices (see Senderoff et. al. (1998) J. Pharm. Sci. 87:183, the entire teachings of which are incorporated herein by reference). The dissolution of insoluble peptide structure in aqueous base is consistent with the breakdown of the intramolecular and intermolecular interactions responsible for beta sheet formation (denatured). Moreover, the isolation of the more soluble form of peptide structure from these solutions is consistent with the reformation of the secondary, alpha helical structure of soluble peptide structure. It is believed that the methodologies of the present invention provide peptide products with significantly reduced aggregation without undue peptide degradation or racemization.

A variety of testing methodologies may be used to qualitatively and/or quantitatively evaluate the deaggregation (solubility) and/or aggregation (insolubility) characteristics of a peptide sample. These methodologies can be used to determine whether a peptide sample should be subjected to a deaggregation treatment in accordance with the invention. These methodologies may also be used after the treatment to assess the peptide quality. The methodologies may be used in the lab or for quality control for pilot plant or commercial scale production of peptides. Indeed, a peptide can be tested at any point in the process of peptide preparation and subject to chromogenic dye testing using the methods as described herein. “Peptide preparation” generally encompasses all the steps that are performed to synthesize a peptide and put it into a desired “end-product” form. These steps can include preparation of the polypeptide chain by addition of amino acid residues using solid phase synthesis, coupling of peptide fragments and/or amino acids using solution phase synthesis, and other steps involved in preparation of the polypeptide chain such as protection, deprotection, precipitation, filtration, extraction, and drying; processes that can be performed (if desired) after the formation of a full-length peptide such as purification, deaggregation, refolding, modification, drying, compounding, and the like.

For example, a simple dissolution test can be used to evaluate the solubility characteristics of a peptide sample. The test is conveniently carried out at a temperature in the range of from about 20° C. to about 27° C., preferably about 25° C. From about 0.1 to about 0.3 grams of peptide is combined with about 1 to about 3 ml of an aqueous medium. The medium may be deionized water. In the case of T-20 or T-1249, these peptides are mildly acetic. Consequently, the medium optionally may include some initial amount base, e.g., about 17 mM Na₂CO₃ or the like, to provide a suitable initial pH. Starting at about pH 6.5 to 7.2, preferably about 6.9 to about 7.0, the pH is incrementally increased, e.g., by 0.2 pH units, using 0.1 N to 1 N NaOH until the peptide is fully dissolved or the pH reaches 9. The mixture is rapidly stirred for 5 to 10 minutes between additional increments. Solids adhering to the sides of the testing vessel should be re-suspended. Rapid mixing should be maintained throughout. Results include observations regarding the clarity of the mixture, time required for dissolution, concentration and amount of caustic used, final pH upon dissolution (if this occurs below pH 9), and the like.

For purposes of the invention, peptides such as T-20, T-1249, and GLP-1, as well as counterparts of these, may be deemed to be in a more desired, deaggregated form if dissolution in accordance with this test occurs at a pH of less than about 8, preferably less than about 7.5. If dissolution occurs at a pH of greater than about 7.5, more preferably greater than about 8, the peptide is deemed to be in a less desired aggregated form. This helps to ensure the practicality of handling the peptide during subsequent processing and handling.

Infrared spectroscopy, particularly fourier transform infrared (FTIR) spectroscopy, is another useful evaluation tool to qualitatively and quantitatively assess the deaggregated or aggregated status of a peptide sample. Generally, certain absorbence peaks of a spectrum are associated with the presence of beta sheet structure, while other absorbance peaks are associated with the presence of alpha helical structure. The relative area of these respective peaks is indicative of the relative amounts of beta sheet and alpha helix structures of the peptide. Generally, the greater the area of the alpha helix peak(s) relative to the area of the beta sheet structure peaks, the more soluble the sample tends to be at physiological pH. Even the soluble embodiments of peptides may incorporate some beta sheet structure content, but the content is sufficiently low such that the soluble peptide embodiments are soluble in aqueous solution at physiological pH. Thus, although practical implementation of the present invention may not eliminate all beta sheet structure from a peptide, the amount of beta sheet structure may be sufficiently reduced so as not to have an undue impact upon the dissolution and biological activity of the peptide.

The use of FTIR techniques to evaluate the beta sheet and alpha helix structure of peptide materials is further described in WO 01/55213, which is incorporated herein by reference in its entirety. Circular dichroism is another technique that may be used.

A problem with FTIR, circular dichroism and similar testing is that these are not specific to beta sheet structure. However, colorimetric analysis does provide such specificity. Colorimetric analysis is another very useful and relatively inexpensive technique that can be used to qualitatively and/or quantitatively assess whether and to what degree the structure of a peptide is aggregated. Some colorants, such as Congo Red or the like, have a very high specificity for binding or otherwise interacting with the beta sheet structure form of peptides but not with the alpha helix structure form. Thus, a peptide sample can be added to a mixture containing the colorant and the visually discernible, colorimetric response to the addition of the peptide is an accurate indicator of undue aggregation. When the peptide contains undue amounts of the aggregated form of the peptide, the color change of the solution is marked and relatively rapid. A cloudy supernatant and precipitate is often observed. For instance, when aggregated T-1249 peptide is added to a Congo Red solution, the initially orange red/colored solution rapidly turns a deep salmon-pink color. Turbidity and precipitation may also be observed in the supernatant. In contrast, when a soluble form of the peptide is added, the color of the solution remains substantially unchanged, and the peptide readily dissolves.

Thus, colorimetric testing is an excellent qualitative indicator for assessing the solubility characteristics of a peptide. The technique is a highly accurate quantitative tool as well. The absorbence spectrum of a calorimetric mixture containing a peptide can be compared to the absorbence spectra of an otherwise identical reference sample having no peptide. If the aggregated β-sheet structure form is present, the sample spectra may tend to be shifted relative to the reference spectra. The differences and/or ratios among the two or more spectra at one or more wavelengths, for example, can be used to quantify the degree of aggregation. For example if the difference between two corresponding peaks is too great, the sample can be deemed to contain too much of the aggregated from. If the difference is below a certain threshold, the sample can be deemed to be sufficiently soluble and/or deaggregated to pass the test. This technique is consistent, reliable, and accurate and, consequently, provides an excellent way to monitor the quality of a peptide product during the time of manufacture and/or at the time of intended use.

Because the degree of beta sheet aggregation correlates to the ease, or lack of ease if the aggregation is too great, by which a peptide formulates and/or readily dissolves in aqueous solution at physiological pH, colorimetric analysis provides a facile and relatively inexpensive way for determining if a peptide batch is suitable for introduction into a subsequent step in peptide preparation or suitable for introducing into a final formulation. The methods allow one to determine the amount of beta sheet aggregation, if present in a sample, and if the amount of such aggregation would allow the peptide batch to be utilized in subsequent steps or formulations.

Peptide with undue amounts of beta sheet aggregation can be problematic at many stages in peptide preparation, formulation, and use. For instance, at the time of use or final formulation, it may be desired to dissolve the peptide in aqueous media typically have a physiological pH. The peptide, though, may be too insoluble, and hence not useable and/or sufficiently active if too much beta sheet aggregation is present. Additionally, beta sheet aggregation can be problematic during manufacture. For example, aggregation can negatively affect filtration by clogging filters, leading to reduced filtration rates or no filtration at all. Aggregation can also affect the appearance or pH of the solution after dissolution, and potentially the stability of the peptide as well.

Preferred colorimetric reagents of the invention generally comprise aqueous media at physiological pH that incorporate one or more water soluble, chromogenic dyes that tend to have greater specificity to associate with beta sheet structure of a peptide as compared to alpha helical structure. This results in a color change and spectral shift in the reagent that depends on the presence and amount of beta sheet structure. That is, the chromogenic dye tends to interact or bind to a more beta sheet aggregated form more readily than a form having lesser beta sheet content. Preferably, the color change and spectral shift quantitatively correlates to the-degree of beta sheet aggregation in a sample. Additionally, the color change or spectral shift that occurs on binding preferably is significant enough to distinguish against background, a sample that is acceptable, or other suitable reference. In other words, it is preferable to use dyes that produce a qualitative and quantitative result showing a clear difference between unacceptable samples and acceptable samples.

One example of a class of suitable dyes is the azo dyes. Azo dyes contain at least one azo group (—N═N—), and typically from one to four azo groups. In one preferred embodiment, the chromogenic dye is a polyazo dye (e.g., includes at least two azo groups such as a diazo dye). The azo dye can also include acidic groups. In some embodiments a preferred acid group is a napthalenesulfonic acid group. A preferred azo dye is selected from the group of Congo Red, Direct Red, derivatives of these dyes, similar dyes, combinations of these, and the like. The azo dye preferably has an absorption maximum in the range of about 475 nm to about 500 nm (in an unbound form). Other contemplated dyes that are similar to Congo Red include Trypan Blue, Direct Red, derivatives thereof, and the like.

The concentration of dye included in the colorimetric reagent can vary over a wide range and may be based on a number of factors, for example, the behavior of the peptide in solution, the amount of chromogenic dye to be combined with the peptide, the desired intensity of the color produced upon association of the dye with an aggregated form of the peptide, the type of chromogenic dye used, and so forth. One of skill in the art will be able to determine the proper concentration of chromogenic dye to be combined upon review of this disclosure. If the concentration is too low, the colorimetric response may show poor resolution among peptide forms. If too high in those preferred embodiments in which quantitative information is desired, the spectral response may not be reasonably linear or otherwise quantitatively correlated to the amount of beta sheet aggregation. As general guidelines, using dyes at concentrations in the range of from about 10 μM to about 100 μm, preferably 20 μM to 100 μm, more preferably 20 μM to about 50 μM is preferred. For instance, in the colorimetric study of T-1249 using Congo Red solutions, the absorbance of Congo Red showed the best correlation to beta sheet aggregation at concentrations up to about 30 μM for peptide concentrations at 0.5 mg/mL or below. An exemplary amount of Congo Red used in calorimetric testing is about 25 μM.

The amount of protein or peptide used should provide a color shift that is detectable, depending on what type of detection method is used. As general guidelines, for example, peptide or protein may be used at concentrations in the range of from about 0.1 mg/mL to about 1.5 mg/mL, more preferably from about 0.1 mg/mL to about 0.5 mg/mL. As one example, calorimetric testing of T-1249 at peptide concentrations in the range of 0.1 mg/mL up to about 1 or 1.5 mg/mL was suitable.

In addition to water, peptide or protein, and the dye, a colorimetric reagent of the invention may further incorporate one or more other ingredients including one or more buffers, and the like. One or more buffers, for instance, can help maintain appropriate pH conditions. Preferred buffers include one or more phosphate-based buffers, such as sodium phosphate. Other suitable buffers are well known in the art and include potassium phosphate, sodium carbonate, sodium bicarbonate, sodium citrate, sodium acetate, ammonium acetate, and sodium acetate. Suitable buffers are those that are substantially non-reactive with the chromogenic dye under the assay conditions used. Suitable salt concentrations would be low enough to avoid salting out the peptide. By way of example, using sodium phosphate at a concentration of about 50 mM was suitable for a Congo Red reagent containing 25 μM Congo Red.

Generally, the colorimetric reagent has a pH that allows an accurate assessment of the behavior of the peptide in conditions that do not promote further aggregation or deaggregation of the peptide or otherwise unduly alter the character of the sample. It is preferred to sample the peptide in a colorimetric reagent having a pH in the range of about pH 6 to about pH 8. These pH ranges are exemplary and can be used for colorimetric analysis of many peptides, including T-1249 and T-1 249-like peptides.

However, other peptides that can be assayed using the chromogenic dye staining methods of the invention may contain a relatively high percentage of acidic residues, such as glutamic acid and/or aspartic acid residues, or a relatively high percentage of basic residues, such as lysine and/or arginine. The pKas of these acidic or basic peptides may cause them to behave differently than peptides having pKas in a neutral pH range. In these cases, the pH of the staining solution can be adjusted accordingly.

To carry out calorimetric analysis, a peptide sample is combined with the colorimetric reagent. The peptide sample can be combined with the chromogenic dye in any suitable manner. In some cases, the peptide and dye can be combined in a well, such as a well in a sample dish, or a container, such as a reaction tube. The material that is used to form the well or container can include plastic, glass, ceramic, or metal, and is preferably non-reactive with the peptide or dye components. Particularly useful wells or containers are fabricated from plastic. The material can optionally be blocked with a reagent, for example a non-aggregated protein or peptide or other suitable blocking reagent, that prevents interaction of the peptide or dye with the material of the well or container. In the case where multiple peptide samples are being tested, multi-well testing plates, such as 12-well, 96-well, or 384-well plates can be used. These multi-well plates can be used to visually observe the chromogenic dye staining and can also be used in connection with continuous wave spectrometers that are able to individually determine the absorbance of the mixture at a particular wavelength. These multi-well plate readers are commercially available from, for example, Amersham Biosciences/GE Healthcare or Titertek (Huntsville, Ala.).

The peptide and chromogenic dye can also be combined on a two-dimensional surface, such as a plastic sheet or film. This can be useful if it is desired to keep the volume of the mixture small. The mixture may be a drop of liquid of, for example, less than 50 μL, wherein the drop is held in a meniscus on the two-dimensional sheet. The drop on the two dimensional sheet may be placed in a humid environment to minimize evaporation of the drop.

Preferably, the mixture is incubated for a period of time and at a suitable temperature to allow the dye and peptide to interact. Representative incubation periods are in the range of from about 5 minutes to 8 hours or more, more preferably 30 minutes to 3 hours. Incubation may occur at a wide range of temperatures such as a temperature in the range of 10° C. to 30° C., preferably 20° C. to about 27° C. By way of example, incubating a Congo Red mixture containing T-1249 for 2 hours at ambient temperature (about 25° C.) would be suitable. The combining and incubation may occur with mixing such as by sonication.

After incubation, the colorimetric response of the reagent may then be visually and/or spectroscopically assessed. For qualitative assessment of color change, the sample mixture and optionally a reference mixture can be transferred to a medium for observation. Visual observation can be aided or enhanced, for instance, by transferring the mixture to a white backing to optimally determine any color change. In some cases the mixture can be spotted and absorbed into a white filter or blotting paper (for example, a cellulose-based paper).

A control or reference sample can be provided by the colorimetric reagent having no peptide. Measurements obtained from control samples can be taken and used in later comparisons against subsequent samples tested for aggregation and/or beta structure content.

For example, control samples can be prepared by mixing Congo Red with a peptide in non-aggregated form, or by preparing Congo Red in solution without any peptide. The color of the Congo Red solution, wherein aggregate binding does not occur, is orange-red and has a maximum absorbance in the range of 485 to 490 nm. Upon the Congo Red binding to peptide in aggregated form, the color of the solution will change to a salmon-pink color, and the mixture will have a maximum absorbance at a higher wavelength (generally about 505 nm and greater).

When an appropriate dye is used, the color of the mixture after incubation will be based on the presence and amount of beta sheet content in the sample. If there is no or very little aggregated peptide in the sample, the mixture will have generally the same color as the reagent per se. If a significant amount of beta sheet content is in the sample, the color of the mixture will appear primarily as the color of the bound chromogenic dye. The peptide may also be precipitated and the reagent supernatant may be turbid. The color difference can be visually observed, for example, without using electronic instrumentation. Visible observation can provide a rapid, qualitative method for determining if peptide batches are suitable or unsuitable for a subsequent use, such as final formulation in aqueous media at physiological pH or the like. For example, after the incubation period with the chromogenic dye, a peptide sample may be observed to exhibit a significant color change, a small color change, or generally no color change at all. A decision can then be made as how to proceed with the subsequent use of the sample. For example, if there is no undue shift in color, these samples may be considered to pass a quality control test. On the other hand, if the color change is too significant, the sample can be deemed to fail the test. In this case, the sample may be subjected to a deaggregation process as described herein to convert the peptide to a more suitable, more deaggregated form.

In yet other cases, the color change may reveal that there is some aggregation, but it may not be clear, based on visual observation, as to whether the amount of aggregated peptide in the batch is sufficiently low enough to be acceptable. Depending on the type of chromogenic dye used, a visual standard chart can be prepared and used to quickly compare the intensity of the staining with a standard if there is a question of whether the sample is suitable or not. In this case the color that is visualized can be compared to a color chart showing the progression of colors from “no aggregation” to “complete aggregation.” The color chart can be based on previous results where batches have been sampled to determine the suitability of a peptide preparation. Furthermore, the color chart can be coordinated with the results from other forms of analysis, as described herein, to provide a quantitative analysis of the peptide batch.

Visual staining can be used in conjunction with, and compared to the results obtained using these electronic methods, including spectroscopy techniques. Simple electronic methods include continuous wave spectrometers, such as optical or UV spectrometry, will be based on the types of dyes that are used and the absorbance when bound to peptide aggregates. Use of spectrophotometers can allow for precise measurements of absorbance at one or more specific wavelengths.

In order to determine the spectroscopic characteristics of a sample at a particular wavelength, the peptide sample that has been stained with a chromogenic dye can be analyzed by UV and/or visible light spectrometry. Results can be read at one or more wavelengths, including the absorption wavelength of the unbound chromogenic dye, and the absorption wavelength of the chromogenic dye bound to peptide aggregate. The spectral response of a sample can be compared to that of a reference to qualitatively and quantitatively correlate to the presence and amount of beta sheet content in the sample. The spectral characteristics can be measured in absorbance units (AU) or milli absorbance units (mAU; 10⁻³ AU). An absorbance unit is a logarithmic unit used to measure optical density, the absorbance of light transmitted through a partially absorbing substance. If T is the percentage of light transmitted, then the absorbance is defined to be −log¹⁰ T absorbance units. An increase in absorbance of 1.0 AU corresponds to a reduction in transmittance by a factor of 10. If the absorbance is 1.0 AU then 10% of the light is transmitted; at 2.0 AU only 1% of the light is transmitted, and so on.

Thus, colorimetric testing can be used as a quality control for determining whether a peptide preparation can be suitable for subsequent processing steps based on the amount of aggregated peptide present in the sample. The method involves determining the spectral characteristics of the colorimetric reagent without peptide as a reference. The baseline absorbance measurement preferably includes (for example in AU or mAU) a wavelength at or near maximum absorbance for the chromogenic dye-peptide aggregate. For instance, obtaining a spectrum over a wavelength range from about 300 nm to about 700 nm would be suitable. Next, one or more peptide samples are mixed with the reagent and absorbance spectra are taken at the wavelength(s) of interest. The peptide samples will typically display measurements that shifted from baseline reading to some degree depending upon beta sheet content of the sample. The spectral shift tends to be greater with increasing beta sheet content. At particular wavelengths, the shift can be compared to the reference spectra, allowing a difference to be calculated. A percentage shift can also be calculated to quantify the spectral change. For example, if the baseline value of the reference reagent at a particular wavelength is 1.000 AU, and three peptide samples are measured to give values of 1.006, 1.055, and 1.033, respectively, at the same wavelength, these samples are within 0.6%, 5.5%, and 3.3% of the baseline value, respectively. The calculated differences would be 0.006, 0.055, and 0.033. respectively. According to the present invention, such shifts can be used to determine if that sample passes a quality control specification. If the peptide sample is deemed to fail the test, it may be desirable to either discard the batch or to subject it to a deaggregation treatment so as to convert the peptide to a form that passes the specification.

The colorimetric results can also be compared to results obtained from other spectroscopic methods including Fourier Transform Infrared (FTIR) spectroscopy, vibrational spectroscopy, or yet other methods such as light scattering (LS), X-ray crystallography, nuclear magnetic resonance (NMR) imaging, circular dichroism (CD), atomic force microscopy (AFM), and cryo-electron micrography. Such techniques can show spectra indicating, for example, the presence of a peptide structure that correlates with an aggregated form, or, for example, the actual observation of peptide particle aggregates (typically using microscopy techniques). In order to correlate results from these techniques with that of the results from the chromogenic dye staining, acceptable and non-acceptable peptide batch samples can be taken and tested in parallel to provide correlative data which can be used to determine peptide batch quality.

Molecular weight analysis is also a useful technique to assess beta sheet structure in a peptide. Depending upon the technique and equipment used, the molecular weight may be the weight average molecular weight, number average molecular weight, actual molecular weight, or average molecular weight. Assessment of average molecular weight or weight average molecular weight are preferred. A peptide formed from a known sequence and number of amino acids generally has a theoretical molecular weight that is known with a fair degree of certainty. The theoretical molecular weight can be calculated from the theoretical peptide structure. The molecular weight of a peptide sample can be determined through any suitable technique such as light scattering analysis. However, the amount of beta sheet structure of a sample impacts its measured molecular weight. Generally, the measured molecular weight tends to be higher with increasing amounts of beta sheet structure being present.

As an example, the T-1249 peptide has a theoretical molecular weight of about 5000. The measured, apparent molecular weight of actual samples of the peptide may range from 6000 to 100,000 or even more. Generally, it has been observed that peptide samples having relatively minor amounts of structure aggregation, e.g., samples showing a molecular weight of about 15,000 (i.e., about 2.5 times the theoretical molecular weight of T-1249) or less, tend to be readily soluble in aqueous solution at physiological pH. In contrast, peptide samples having a molecular weight of more than about 10,000 and often more than 20,000 tend to less soluble. Samples showing a molecular weight on the order of 40,000 or more are highly insoluble and thus believed to be highly aggregated and containing significant amounts of beta sheet structure.

Molecular weight analysis can be used in connection with chromogenic dye analysis. Light scattering will show that if beta sheet aggregates exist, the molar mass will be higher, and typically significantly higher, than the theoretical molar mass of the peptide. According to the invention, it has been shown that an increase in the molecular mass of the peptide sample correlates with an increase in chromogenic dye staining, that is, an increase in the absorption at the wavelength which is specific for the chromogenic dye-peptide complex.

According to a preferred methodology of the present invention for carrying out a deaggregation treatment, a sample comprising the peptide is dissolved in an aqueous, buffered, alkaline solution. In some instances, the peptide is provided as a dried, purified powder, such as in the form of a dried powder purified using HPLC techniques. Optionally, the powder has been lyophilized. Agitation may be used to assist with the dissolution of the peptide in the solution, although agitation should not be so vigorous so as to unduly risk damaging the peptide material.

The aqueous, buffered, alkaline solution is generally derived from ingredients comprising water, at least one salt, and a sufficient amount of at least one base to provide the desired dissolution pH. The peptide and various ingredients constituting the aqueous, buffered, alkaline solution may be combined in any order. In one mode of practice, the solution is prepared from its constituent ingredients and then the peptide is added to the already prepared solution. In another mode of practice, the peptide may be added to an aqueous solution comprising the salt wherein the solution has a pH that is too low for dissolution to occur. A base is then added to this mixture in order to raise the pH to a value at which dissolution will occur. As still yet another alternative, the salt may be added to the solution before, during, and/or after dissolution. Generally, though, the salt is incorporated into the solution before the pH is lowered in a manner to cause the peptide to precipitate as is described further below.

The concentration of the peptide in the solution may vary over a wide range. This concentration depends upon a peptide's characteristics and may be determined using routine experimentation. As general guidelines, the peptide concentration in the solution may be in the range of from about 3 to about 6 g/l. In a specific mode of practice, preparing a solution containing about 4 g/l of T-1249 peptide was found to be suitable. As used herein, the concentration of this and other ingredients included in the solution, unless otherwise expressly noted, is determined based upon the volume of the solution at the time the ingredient is added to the solution. Thus, the volume of solution for purposes of determining ingredient concentration would not include co-solvent (described below) if the co-solvent is added later, as is preferred.

A variety of one or more bases may be incorporated into the solution to provide the desired pH. Representative examples of suitable bases include hydroxide bases such as NaOH and bicarbonate and carbonate bases such as sodium or potassium bicarbonate or sodium or potassium carbonate. Sodium hydroxide is preferred, especially 0.5 N to 1 N NaOH. The base is used to adjust the pH to a desired value at which the peptide will dissolve in the solution in a reasonable amount of time. The desired pH value will vary depending upon the nature of the peptide. As general guidelines, if the pH is too low, then dissolution may be partial or may not occur to any significant degree. Even if dissolution were to occur, dissolution might take too long if the pH is too low. On the other hand, if the pH is too high, dissolution may readily occur, but the peptide could be damaged. Balancing these concerns, the pH desirably is as low as might be practical to achieve dissolution in a time frame of from about 1 second to about 3 hours, more preferably less than 2 hours. For many peptides, this corresponds to a dissolution pH in the range of from about 8 to about 11. In a specific mode of practice, dissolving T-1249 peptide in a solution at a pH of about 10 has been found to be suitable.

The salt constituent(s) of the solution improve the dissolution characteristics of the resulting precipitated peptide. Specifically, a soluble peptide that dissolves readily in aqueous solution at lower pH is prepared more consistently when a salt is present at an appropriate concentration. In the absence a salt, the resultant peptide may be difficult to dissolve at physiological pH and, indeed, may only dissolve at unduly higher pH, e.g., 8 or higher, often 9 or higher, and even 11 or higher. Without wishing to be bound, it is believed that the presence of the salt assists in the precipitation of peptide with a more desired, substantially reduced amount of aggregation. In contrast, in the absence of the salt, the peptide may have a tendency to precipitate in a more aggregated, and less desirable, form.

The fact that the salt helps prevent aggregation upon precipitation of a denatured peptide is counter-intuitive to some degree. Generally, salt might be expected to cause aggregation, because the resultant higher ionic strength of the mixture will tend to compress the ionic sphere (also known as the ionic double layer) about each peptide molecule. This allows peptide molecules to come closer together, making it more likely for shorter range, aggregate-inducing forces to come into play. This phenomena is routinely used to precipitate peptide and is known as salting out. However, the salt of the present invention is used at relatively low concentrations at which the salting out phenomena does not occur. Instead, it is believed that the relatively low concentration of salt helps to solvate the ionic groups on a peptide and thus help bring it into solution.

A variety of salts would be useful in the practice of the present invention. Examples include sodium carbonate, sodium acetate, ammonium carbonate, ammonium acetate, sodium bicarbonate, ammonium bicarbonate, sodium and potassium versions of these, combinations of these, and the like. Ammonium acetate is most preferred as use of this salt consistently yields precipitated peptide with excellent dissolution characteristics.

The concentration of the salt in the solution may vary over a wide range. Generally, salt is added in an amount that helps protect the secondary peptide structure in the dissolved state and during precipitation. Suitable concentrations may be enough to provide some, little, or generally no buffering action. If too little salt is used, the resultant peptide may show poor dissolution characteristics in aqueous solution at physiological pH. On the other hand, if too much salt is used, then the solubility tends to decrease, and the peptide could precipitate prematurely. Additionally, the peptide may not filter easily after precipitation. Balancing these concerns, using 1 to 200 mM equivalents of salt is one example of a salt concentration range that would be suitable in the practice of the present invention. In a specific mode of practice, using about 5 mM to about 50 mM, more preferably about 10 mM equivalents of salt, especially ammonium acetate, has been found to be suitable.

The dissolution temperature(s) generally refers to the temperature(s) of the aqueous, solution in which the peptide is dissolved. Dissolution may occur at any suitable temperature. The desired dissolution temperature will depend to a large degree upon the nature of the peptide being dissolved. If the temperature of the solution, though, is too low, it may be more difficult to achieve dissolution in a reasonable amount of time. If the temperature is too high, the peptide could be damaged and/or the dissolution characteristics of the resulting precipitated peptide may be poor in aqueous solution at physiological pH. Generally, dissolving the peptide in a solution maintained at one or more temperatures in a range from about 10° C. to about 30° C., preferably about 10° C. to about 25° C., more preferably about 15° C. to about 20° C. would be preferable. In specific modes of practice, carrying out dissolution at 16° C., 20° C., and 25° C. has been suitable.

A co-solvent is preferably incorporated into the solution so that subsequent precipitation of the peptide occurs in the presence of the co-solvent. The co-solvent can be added to the solution before, during, and or after dissolution, but preferably is added promptly after dissolution of the peptide. The co-solvent refers to one or more additional solvents in which the peptide is soluble at the dissolution pH. Preferably, the peptide is also soluble in the co-solvent at 25° C. and physiological pH when the peptide is sufficiently deaggregated that ratio of the measured molecular weight of the peptide to the theoretical molecular weight of the peptide is in the range from about 2:1 to about 1:1. Examples of co-solvents include acetonitrile, methanol, combinations of these, and the like. Acetonitrile is preferred, particularly when the peptide is T-1249.

The presence of the co-solvent favorably impacts the dissolution characteristics of the resultant peptide. Specifically, this approach helps to more consistently provide a peptide product that readily dissolves in aqueous solution at physiological pH. In the absence of a co-solvent, the peptide product may dissolve more slowly and/or only dissolve completely at a pH that is higher than is desired. Without wishing to be bound, it appears that the co-solvent facilitates precipitation of peptide in a substantially more deaggregated form than might otherwise occur if no co-solvent is present.

In preferred embodiments of the invention, a sufficient amount of co-solvent is added to the solution such that the solution contains from about 2 to 50 volume percent, preferably from about 5 to about 30 volume percent, and more preferably from about 10 to about 20 volume percent of the co-solvent. The amount of co-solvent incorporated into the solution, however, can impact the solubility characteristics of the resultant peptide, although the amount of co-solvent needed to obtain more preferred results depend upon factors such as the nature of the peptide, the temperature at which precipitation is carried out, the rate at which precipitation is carried out, and the like.

For instance, when no co-solvent was used, it was found that the resultant T-1249 particles dissolved very slowly with high turbidity even after 14 to 18 hours and even at a pH of 8. A co-solvent improved this performance, although too much or too little provided less than optimum results. With 14% by volume acetonitrile, the peptide product dissolved to some degree at a pH of 6.95 but not completely. The dissolution behavior improved significantly with 18% by volume acetonitrile, as the product dissolved rapidly in 1 hour to a final pH of 6.69 with only very faint turbidity. Yet, when the acetonitrile concentration was increased to 20 to 22 volume percent in additional tests, the dissolution pH was higher, generally around 9.

After dissolution, and desirably after addition of the co-solvent, the pH of the solution optionally further is increased by adding additional base in order to facilitate further deaggregation of the peptide, if desired. For instance, in the case of T-1249, the pH may be increased to pH 11 by adding additional 1 N NaOH. The solution is then desirably promptly filtered. Pressure filtering through a 0.2 micron filter would be suitable. The filtrate is optionally degassed under vacuum, after which the solution may be aged for a suitable time period before further processing in order to complete the deaggregation process. The desired amount of aging, if any, will depend upon factors such as the nature of the peptide, the dissolution temperature, the nature of the co-solvent, and the like. Generally, aging so that the total time that the peptide is at the elevated pH (including not just aging time, but also filtering time, degassing time, etc.) is in the range of from about 5 minutes to about 6 hours, more preferably about 30 minutes to about 2 hours. After aging, the solution optionally may be filtered again.

After aging, the pH of the solution is reduced, e.g., acidified, under conditions effective to cause the peptide to precipitate. According to the principles of the invention, this is accomplished so that the resultant peptide is readily soluble in aqueous solution at physiological pH. Without wishing to be bound, it is believed that the methodology reduces the tendency of the peptide to precipitate in a form containing undue aggregation of the peptide. The desired final pH will vary depending upon the peptide, but typically will be low enough to cause precipitation of the peptide at a desirable high yield without having the pH be so low as to risk damaging the peptide. As general guidelines, a final pH in the range of from about 3 to about 6, preferably 4 to about 6 could be suitable. As a specific example, a final pH of 5.3 to 5.5 is desirable with respect to the T-1249 peptide.

The pH of the solution preferably is lowered by adding one or more acids to the solution. Examples of acids include HCl, sulfuric acid, acetic acid, oxalic acid, combinations of these, and the like. Acetic acid is preferred. For instance, aqueous, 5% or 10% acetic acid solution have been found to be suitable.

The time period over which the pH is lowered can impact the dissolution characteristics of the resulting precipitated peptide. If this time period is too slow, e.g., the acid is added at too slow a rate, the resultant peptide may be dissolve too slowly and/or be insufficiently soluble at physiological pH. It is believed, therefore, that the peptide tends to precipitate in a form with undue amounts of aggregation if pH lowering takes too long. Yet, rapidly lowering the pH all the way to the final pH is not required in all instances. In some modes of practice, peptide product with excellent dissolution properties can still be obtained if the acid is added relatively rapidly to lower the pH only to an intermediate pH. After this initial, relatively rapid addition of acid, acid is added at a second, relatively slower rate to lower the pH of the solution to the final desired pH. Using such a multistage approach to lowering the pH allows the pH reduction to occur without undue peptide aggregation while also minimizing the risk of overshooting the final desired pH. Suitable intermediate pH values would be in the range of from about 6 to about 8, more preferably from about 6.0 to about 7.5. Desirably, the initial rapid lowering of the pH occurs in a time period of less than about one hour, preferably 30 minutes or less, more preferably 15 minutes or less.

For example, one suitable mode of practice involves lowering the pH of a T-1249 solution initially at a pH of 11. A sufficient amount of acid is added relatively rapidly over a period of 10 minutes to lower the pH to an intermediate value of about 6.0. Then, acid is added more slowly over 10 to 20 minutes to lower the pH to 5.3 to 5.5.

While not wishing to be bound, a rationale to explain the benefits of rapid acidification can be suggested. When the pH is lowered slowly, the charge associated with a molecule is slowly reduced, and the kinetics of aggregation can occur in solution. When pH is lowered quickly, the peptide precipitates so quickly that there is too little time for undue organization to occur on the molecular scale leading to β-sheet formation.

The mixture is desirably mixed well during the course of adding the acid to cause precipitation of the peptide. Yet, the dissolution quality of the resultant peptide can be compromised if agitation is too aggressive. The filtering characteristics of the peptide also can be compromised if the agitation is too aggressive or too mild. It is believed that excessive shear can lead to “striation” at a molecular scale, stretching out protein molecules and thereby sterically facilitating chain-to-chain interaction and i-sheet aggregation.

By way of example, 0.57 liter of a solution at ambient temperature containing about 2.5 grams of peptide was mechanically mixed during acid addition with a mixing blade having 2 blades and a diameter on the order of about 3 inches. Tests were conducted at 100, 200, 400, and 600 rpm. The peptide obtained at 100 rpm filtered poorly. The peptides obtained at the higher mixing rates filtered better and also dissolved readily at relatively low pH. These laboratory results suggest moderate to high agitation during precipitation is preferable. Further tests, though, showed that foaming during precipitation is undesirable. In one test, the mixture was recirculated above the liquid surface by a diaphragm pump for pH measurement in the recirculation loop. This recirculation above the liquid surface coupled with higher agitation generated a significant amount of foam. The resultant peptide showed poor compounding characteristics.

As general guidelines, therefore, it is preferred to agitate the mixture while adding the acid as vigorously as is practical while leaving a sufficient safety margin to avoid foaming the mixture.

The addition of acid to cause precipitation of the peptide may be carried out with the solution at any suitable temperature. Generally, if the temperature is too hot or too cold, the peptide could degrade or otherwise precipitate with undue aggregation to the undesired structure. As guidelines, carrying out precipitation at a temperature in the range of 10° C. to 30° C., preferably 15° C. to 25° C., most preferably 16° C. to 18° C. would be suitable.

After precipitation, the peptide is desirably isolated and dried before being combined with other ingredients, lyophilized, packaged, stored, further processes, and/or otherwise handled. This may be accomplished in any suitable fashion. According to one suitable approach, the peptide is collected via filtering, washed with ample water washes to reduce final salt content to a suitable level, and then dried. It has been found, however, that the precipitated peptide may show gel-like properties. The gel-like precipitate can be difficult to filter. Consequently, prior to filtering, the precipitate preferably is subjected to an aging process with desirable agitation in which the peptide particles are agglomerated to “harden” the particles. This kind of particle agglomeration constituting particle hardening is different from the undesirable aggregation that leads to beta sheet structure on the one hand or the desired a-helix structure on the other. Specifically, aggregation takes place on a molecular scale, whereas agglomeration takes place in a macroscopic or particular scale. Such aggregation may constitute and/or be similar to quaternary structure, which refers to non-covalent complexes of multiple polypeptide chains with other macromolecules. Agglomerated peptide is more practically handled and isolated.

In a preferred mode of practice, this age-hardening treatment involves aging the peptide with agitation in the course of an innovative cooling/heating/cooling treatment. This improves the filtering characteristics of the peptide without undue damage of the peptide tertiary structure. In a specific mode of practice, the treatment involved aging the particles in aqueous mixture for 5 minutes to 48 hours, preferably 30 minutes to 8 hours, more preferably 30 minutes to 2 hours at a first temperature below ambient temperature preferably being in the range of from more than 0° C. to about 20° C., preferably 10° C. to 20° C., more preferably about 16° C. Agitation desirably is used to ensure that the particles are well dispersed during the aging. Conveniently, the same kinds of agitation conditions may be used for this aging treatment as were used during precipitation.

Next, the temperature of the mixture is increased by about 2° C. to about 30° C., preferably about 5° C. to about 15° C. to a moderately warmer temperature, wherein the transition to the warmer temperature occurs with agitation over a period of from about 1 minute to about 48 hours, preferably 5 minutes to 8 hours, more preferably 20 minutes to 2 hours. Preferably, the new, moderately warmer temperature is still at ambient or below. In a specific mode of practice, increasing the temperature from 16° C. to 21° C. in about one hour was found to be suitable. Agitation desirably continues during this transition. The mixture is then aged at the warmer temperature for a period of from 5 minutes to 8 hours, preferably 20 minutes to 4 hours, more preferably about 3 hours, with agitation.

After this aging step, the temperature of the mixture is lowered by about 2° C. to about 30° C., preferably about 5° C. to about 15° C. to a moderately cooler temperature, wherein the transition to the cooler temperature preferably occurs with agitation over a period of from about 1 minute to about 48 hours, preferably 5 minutes to 8 hours, more preferably 20 minutes to 4 hours. Preferably, the new, moderately cooler temperature is in the range of from above about 3° C. to about 18° C., more preferably about 10° C. In a specific mode of practice, lowering the temperature from 21° C. to 10° C. in about two hours was found to be suitable. The mixture is then further aged at the cooler temperature preferably for a period of from about 5 minutes to 48 hours, more preferably about 6 hours.

This aging treatment improves the filtering characteristics of the precipitated particles in that filtering and separating the peptide particles from the filtrate occur more readily without unduly changing the secondary structure of the peptide. Without wishing to be bound, it is believed that the particles agglomerate by the warming, which causes the peptide particles to become soft (or rubbery) and tacky. The resultant agglomerates are hardened by cooling back down to isolation temperatures. In the rubbery state, very minor amounts of aggregation might occur, albeit slowly.

Thus, after this aging, the precipitate is filtered, preferably pressure filtered such as with 1 psig N₂. The filter cake may be washed one or more times with water desirably pre-cooled such as to a temperature in the range of from about 3° C. to about 20° C., preferably 5° C. to about 15° C., more preferably about 10° C. This helps to lower the salt content of the cake. The filter cake may then be partly or wholly dried, such as by passing nitrogen through the cake with nitrogen at a suitable temperature for a suitable time period, such as 1 minute to 48 hours, preferably 5 minutes to 8 hours, more preferably about 6 hours. Using nitrogen that is at about ambient temperature is convenient and suitable. The cake may be periodically mixed to facilitate drying. Drying optionally may be completed in a separate drying apparatus. Such optional drying preferably occurs under vacuum, e.g., less than 30 mm Hg, at a moderate temperature so as not to degrade the peptide, e.g., at a temperature less than about 30° C., preferably less than about 28° C.

The principles of the present invention will now be further illustrated with respect to the following illustrative examples.

### EXAMPLE 1

The following procedure may be used to subject a peptide sample to a deaggregation treatment in accordance with the present invention.

T1249 peptide(basis: 3 kg) is dissolved in 10 mM aqueous NH₄OAc (708 L) at pH 10 and 16° C. using 0.5N NaOH (˜16 kg). Acetonitrile (ACN) (134.2 kg) is added to the mixture to 18 volume % (v/v). The pH is adjusted to 11 with 0.5N NaOH (˜5.7 kg). The mixture is then pressure filtered through 0.2 μ filter for about 1 hour. The filtrate is degassed using vacuum for a period of about 30 min and then is aged for ½ hour at pH 11. The total time at pH 11 is about 2 hours.

During the aging in step, an appropriate RPM for subsequent precipitation in a 2000 L vessel is determined. Using a 36 inch diameter blade for mixing in a 200 liter vessel, the initial agitation rate is set at 40 RPM. The mixture is stirred in this way for 5-10 min and observed for foam and/or emulsion. This procedure is repeated by increasing the RPM by 5 units until foam and/or emulsion is observed so long as the mixing rate does not exceed 75 RPM. The mixing rate for subsequent precipitation is set at an RPM at 5 units below the RPM at which foam and/or emulsion is observed. If foam and/or emulsion were to be observed at any time during the precipitation, the rpm would be reduced in increments of 5 so long as the rpm is not reduced below 40 RPM.

After aging, precipitation is carried out at 16° C. without recirculating but with agitation at the rpm determined above. As necessary, the RPM is increased to maximum for a minute to knock down the wall cake. Then 12 kg 5% (v/v) aqueous Acetic Acid (AA) is charged in <10 min to a pH of 6.0. 6 kg 5% (v/v) of aqueous AA is then charged in 10-20 min at a slower addition rate to a pH of 5.5. The contents are adjusted to a final pH to 5.3-5.5 quickly (<10 min).

The contents including the precipitated peptide are aged for 1 hour at 16° C. with the same RPM used in the precipitation steps. Then, the contents are heated 21° C. in about 1 hour using a maximum bath temperature of 28° C. The contents are aged for 3 hours at 21° C. using the same RPM used in the precipitation step. The contents are then cooled to 10° C. in about 2 hours (It is recommended not to go below 3° C. for a bath temperature.) and aged for at least 6 h at 10° C. and 40 RPM. This aging treatment would reduce the gel-like characteristic of the initial precipitate and make the precipitate more suitable for subsequent filtering.

After the aging treatment, the contents are filtered using about 1 psig N₂. The filter cake is washed with 150 L high purity (“HP” ) water precooled to 10° C. The cake is blown down for at least 6 hours with N₂ flow and vacuum at ambient temperature. The filter cake is periodically mixed/smoothed during filtering. As needed, the product is further dried in a dryer at <28° C. bath and <30 mm Hg vacuum.

### EXAMPLE 2 (COMPARATIVE)

This comparative example shows how adding acid too slowly during precipitation can lead to a peptide product that is still too aggregated. Sample A was a portion of a highly aggregated batch of T-1249 peptide having an average molecular weight of 98,890±6660. The molar mass was determined using MALS light scattering. Sample A showed a dissolution pH of 8.983. Sample A was subjected to a deaggregation treatment in which acid was added very slowly over a period of 169 minutes. The re-worked Sample A showed a dissolution pH of 7.63 in one analysis and 7.52 in another analysis, showing normal variation among the samples. These dissolution characteristics are moderately too high to be suitable. At dissolution, the mixture was faintly turbid and showed a turbidity of 13 NTU. The molecular weight of the re-worked Sample A using MALS light scattering was about 27,000.

The procedure used to re-work Sample A was as follows. A 2000 L glass-lined vessel equipped with a jacket and temperature control was used as the T1249 dissolution vessel. The vessel was charged with 666 g of Ammonium Acetate and 865 kg High Purity Water (HPW) and stirred to yield 10 mM Ammonium Acetate solution. The dissolution vessel temperature was adjusted to 16±1° C. About 80 L of Ammonium Acetate solution was taken out from the T1249 dissolution vessel and stored in a drum for use as a rinse in a later step (filtration of solution at a pH of 11). 3.665 kg Sample A was charged to the precipitation vessel and stirred. 19.55 kg of 0.5N Sodium Hydroxide (NaOH) solution was slowly charged to the dissolution vessel to dissolve the solids. The pH of the solution at the end of dissolution was 10.0. 164 Kg of acetonitrile (ACN) was charged to the precipitation vessel. 6.95 Kg of 0.5N Sodium Hydroxide (NaOH) solution was charged to the dissolution vessel to raise the pH to 10.92.

The peptide solution was pressure filtered through a 5 μm filter followed by a 0.2 μm filter. The filtrate was collected in a 2000 L glass line precipitation vessel equipped with a jacket, stirrer (36″ diameter retreat curve impeller), temperature control, a pump to re-circulate the vessel contents from the vessel bottom back to the top of the vessel, and pH probe in the recirculation loop. The 10 nM Ammonium Acetate solution (approximately 80 L) drummed in the Ammonium Acetate preparation step was charged to the dissolution vessel as a rinse. The Ammonium Acetate solution was pressure filtered through a 5 μm filter followed by a 0.2 μm filter to the Precipitation vessel.

The precipitation vessel temperature was adjusted to 16±1° C. The total time the peptide solution was at a pH of 10.92 was 129 minutes. 25.4 Kg of 5% Acetic Acid solution was slowly charged to the precipitation vessel in 169 minutes at 80 RPM to a final pH of 5.48. A solution sample taken during the acid addition step at a pH of 9.3 and analyzed for the molar mass by the MALS method. The molar mass was 6500 which indicated that the peptide solution was in a de-aggregated state prior to the precipitation. The T1249 slurry was aged at 16±1° C. for 85 minutes at a reduced RPM of 70. Then the slurry was heated to 21±1° C. in 1 hour and aged for 100 minutes at a reduced RPM of 60. The batch was finally cooled to 10±1° C. in 88 minutes and aged for 7 hours at a reduced RPM of 30.

The product slurry was filtered in a Nutsche filter fitted with a 5-10 μm polypropylene filter cloth using ˜5 psig nitrogen on the cake. The filtration time for the slurry was 1 hour. The filter cake was washed with 150 Kg of HPW and blown down with nitrogen for 4 hours. The wet cake was dried at 21-28° C. under 25 mm Hg vacuum. The weight of the dry product was 3.0 Kg with a moisture content of 6.2% by Karl Fischer (KF) analysis.

The dry product was tested for dissolution with the following results. The dissolution pH was 7.63 with a turbidity of 23 NTU. The dissolution time was 85 minutes. The solution appeared to be faintly turbid. Dissolution pH for another sample of the dry product was 7.52. Clearly the product failed the dissolution test.

### EXAMPLE 3

Sample B was a portion of a highly aggregated batch of T-1249 peptide having an average molecular weight of 49,480±5200. The molar mass was determined using MALS light scattering. Sample B showed a dissolution pH of 9.968. Sample B was subjected to a deaggregation treatment in accordance with the procedure set forth in this example. The resultant re-worked Sample B showed an average molecular weight via MALS of 9700. The re-worked Sample B showed a dissolution pH of 6.798. Upon dissolution, the solution was faintly turbid. The turbidity was only 2.0 NTU. A 2000 liter vessel was used containing a 3 foot diameter agitation blade. The vessel was equipped with temperature and pH probes.

The following general sequence of steps may be carried out to re-work Sample B. Specific conditions actually used to re-work Sample B follow the general procedure.

The following initial steps are used to dissolve Sample B in an aqueous medium:

1. Setup a T1249 dissolution vessel equipped with bath temperature and pH probes.
2. Charge 0.701 kg Ammonium Acetate.
3. Charge 909 kg High Purity Water (HPW) to prepare 10 mM Ammonium Acetate solution and stir to dissolve the solids.
4. Adjust the temperature to 16±1° C.
5. Separately store about 80 L Ammonium Acetate solution for use in step-16 below as rinse during the filtration at pH 11.
6. Charge 4 kg of peptide.
7. Charge 0.5N NaOH slowly to adjust the pH to 10±0.2 and dissolve the peptide. Expected charge is 22 kg.
8. Ensure that the solids are completely dissolved.
9. Charge 158 kg Acetonitrile (ACN).
10. Sample the peptide solution and check for % ACN by GC. Target is 18 v/v % ACN (relative to initial ammonium acetate buffer charge in step-3). Adjust the composition as necessary.
   The following steps were carried out to achieve peptide deaggregation:
11. Adjust the pH to 11±0.2 with 0.5N NaOH (expect ˜6.6 kg charge).
12. Setup the peptide precipitation vessel equipped with a bath. Set the bath temperature at 16±1° C.
13. Age the peptide solution at pH 11 for 1 h. Ensure that the contents are clear without any solids.
14. Pressure filter the peptide solution through a 5 μm filter followed by a 0.2 μm filter and collect the filtrate in a precipitation vessel.
15. Charge the 10 mM Ammonium Acetate solution drummed in step 5 to the dissolution vessel as a rinse.
16. Pressure filter the rinse solution through a 5 μm filter followed by a 0.2 μm filter to the Precipitation vessel.
17. Degas the filtrate using 50-150 mmHg pressure for 15 to 30 minutes at moderate agitation (tip speed of 2.5-3.0 m/s or 50-60 rpm with the 3 ft diameter agitator blade).
18. Adjust the precipitation vessel temperature to 16±1° C.
19. Age for additional time at pH 11 so that the total age time at pH 11 is 3 h (start of step 13 to end of step 19).
   After deaggregation, the following sequence of steps were carried out to cause precipitation of the peptide in a highly soluble form:
20. Adjust the agitation to a tip speed of 2.5 to 3.0 m/s (55-60 rpm with 3 ft diameter agitator blade) to provide turbulent mixing. If necessary, reduce the agitation to avoid excessive foaming.
21. Charge 16 kg (two thirds of the total charge) of 5% aqueous acetic acid in <5 min through a spray nozzle located above the liquid surface approximately half way between the vessel wall and the agitator shaft to a pH of ˜6.0.
22. Charge 8 kg (one third of the total charge) of 5% aqueous acetic acid in 5-10 min to a pH of ˜5.5.
23. Adjust the pH to 5.3-5.5 quickly (<15 min) with 5% aqueous acetic acid.
24. Age the slurry for I h at 16±1° C.
25. Using a maximum jacket temperature of 28° C., heat the slurry to 21±1° C. in ˜1 h.
26. Age the slurry for 3h at 21±1° C.
27. Cool to 10±1° C. in <2 h (use a bath temperature of 3 ° C. at the start).
28. Age for at least 6 h at 10±1° C. at reduced rpm (40 rpm with 3 ft diameter agitator blade or a tip speed of ˜2 m/s).
   The following sequence of steps may be used to isolate the aged, precipitated peptide:
29. Charge ˜500 L deionized water (DIW) to the wash the precipitation vessel and cool the slurry to 10±1° C.
30. Pressure filter the slurry in a Nutsche filter fitted with an 8-10 μm polypropylene filter cloth using ˜1 psig nitrogen on the cake and 500 mmHg vacuum on the filtrate. Leave 1-3 inches of liquid over the cake at the end of filtration.
31. Using slight vacuum (550-600 mmHg) and no pressure, wash the cake with high purity water (2×200 L) precooled to 10±1° C. Monitor the conductivity of the filtrate. If necessary use additional wash to bring down the conductivity of the effluent to <20 microsiemens.
32. Dry the product at ambient temperature (20-25° C.) with high nitrogen flow (5-10 psig nitrogen pressure on cake) through the cake and vacuum from the cake bottom. Stir the cake every 4-6 hours and sample for KF. The target KF is 6-8%.
33. When the product is dry, package into double poly-lined fiber packs.

### EXAMPLE 4

The procedure of Example 3 was used to process two additional peptide samples except as noted in the following table. For convenience, process details of Samples A and B are also provided. The first sample was the re-worked Sample A prepared in Example 2. The resultant treated sample is referred to herein as re-worked Sample A′ The molar mass of re-worked Sample A′ was determined to be about 9300 using MALS light scattering. The re-worked Sample A′ showed a dissolution pH of 6.79. Upon dissolution, the solution was clear. The turbidity was only 2.0 NTU.

The second sample, Sample C, was a portion of a highly aggregated batch of T-1249 peptide that showed a dissolution pH of 9.235. The resultant re-worked Sample C showed a dissolution pH of 6.73. Upon dissolution, the solution was clear. The turbidity was only 3 NTU.

For convenience, process details of re-working Samples A and B are also provided. As used herein, NTU refers to nethelometric turbidity unit. MALS refers to multi-angle light scattering. GC refers to gas chromatography. QC refers to quality control. LOD refers to loss on drying. KF refers to Karl Fischer. NA means not applicable. ND means not determined. NM means not measured. RT means room temperature.

| Re-worked Sample | | | |
|---|---|---|---|
| Sample A | Sample B | Sample A′ | Sample C |

Analytical data of re-worked material after treatment:

| | | | | |
|---|---|---|---|---|
| Dissolution pH | 7.52 | 6.78 | 6.79 | 6.73 |
| Dissolution Appearance/ | VFT/13 | VFT/2.0 | clear/2.0 | Clear/3 |
| Turbidity (NTU) | | | | |
| MALS Ave MW | 27,000 | 9,700 | 9300 | |
| Congo Red 528 Absorbance | 71 | 55 | 52 | |
| (mAU) | | | | |

Compounding data for re-worked material after treatment:

| | | | | |
|---|---|---|---|---|
| Compounding results | FAIL | PASS | PASS | PASS |
| Solution pH/Appearance | 6.86/slightly | 6.4/Clear | 6.6/clear very | |
| | turbid | few fibers | few fibers | |
| Filtration Tuffryn Time (min) | 10% Tuffryn/ | 2.5 | 1.75 | |
| | 3 nylon filters | | | |
| Filtrate Turbidity (NTU) | 11.3 | 1.6 | 1 | |

Deaggregation data

| | | | | |
|---|---|---|---|---|
| Ammonium Acetate charge, g | 666 | 805 | 490.7 | 771.1 |
| Amm Acetate, mM | 9.99 | 9.97 | 10.03 | <10 |
| HPW, kg | 865 | 1048 | 635 | 1090 |
| T-1249 charged, kg | 3.665 | 4.6 | 2.77 | 4.83 |
| T-1249 concentration, g/L | 4.24 | 4.39 | 4.36 | 4.43 |
| 0.5 N sodium hydroxide used, kg | 19.55 | 25.2 | 15.7 | 22.4 |
| Final pH | 10 | 10 | 10.2 | 9.84 |
| Temperature during pH 10 | 16 | 16 | 16 | 16 |
| adjustment, ° C. | | | | |
| Agitator speed during pH 10 | 50 | 50 | 50 | 50 |
| adjustment, RPM | | | | |
| time to pH 10, min | 27 | 16 | 4 | 30 |
| Time batch was at pH 10, hr:min | 2:15 | 2:17 | 3:00 | 2:00 |
| ACN charged, kg | 164 | 179.9 | 111.2 | 187.5 |
| ACN concentration, calculated | 15.92% | 15.30% | 16.30% | |
| W % | | | | |
| ACN concentration, calculated | 19.5% | 18.0% | 18.3% | |
| v/v % | | | | |
| ACN concentration, by GC | 19.3% v/v | 15.2 w/w % | 15.50% | 16% |
| 0.5 N sodium hydroxide used, kg | 6.95 | 7.6 | 3.8 | 9.5 |
| Final pH, QC | 10.92 | 10.9 | 11.1 | 10.98 |
| Temperature during pH 11 | 16 | 16 | 16 | 16 |
| adjustment, ° C. | | | | |
| Agitator speed during pH 11 | 50 | 50 | 50 | 50 |
| adjustment, RPM | | | | |
| time to pH 11, min | 17 | 3 | 2 | 60 |
| 0.2μ filtration time, hr:min | 1:18 | 1:00 | 0:24 | 0:23 |
| Filtration pressure, psig | 7 | 12 | 3 | 20 |
| Flow rate, kg/min | 20-22 | 20 | 20 | |
| Time batch was at pH 11, hr:min | 2:09 | 2:00 | 4:00 | 2:00 |
| Degassed time, min | 0 | 15 | 15 | 15 |

Precipitation data:

| | | | | |
|---|---|---|---|---|
| Precipitator vessel volume, L | 2000 | 2000 | 2000 | 2000 |
| Recirculation on/off | on | off | off | off |
| Recirculation dipleg submerged? | yes | NA | NA | NA |
| Foam visible? | yes | yes | slight | slight |
| 5% acetic acid used, kg | 25.4 | 30.6 | 17.9 | 30.5 |
| Time to add 90-95% of the acid, | >120 | 15 | 17 | 10 |
| min | | | | |
| Time to add remaining acid, min | | 30 | 10 | 10 |
| Batch temperature during acid | 16 | 16 | 16 | 16 |
| addition | | | | |
| Agitator speed during acid | 80 | 60 | 55 | 60 |
| addition, RPM | | | | |
| Tip Speed, m/s | 3.8 | | | 2.85 |
| Final pH, QC | 5.48 | 5.41 | 5.42 | 5.43 |
| Time to pH 9.3 | | ND | ND | ND |
| Hold time pH 9.3, min | 15 | 0 | 0 | 0 |
| total acid addition time, min | 169 | 45 | 27 | 20 |
| Age temperature, ° C. | 16 | 16 | 16 | 16 |
| Age time, hr:min | 1:25 | 1:00 | 1:00 | 1:00 |
| Age agitator speed, RPM | 70 | 55 | 55 | 55 |
| Heat up time, hr:min | 1:00 | 1:00 | 0:49 | 1:00 |
| Heat up target temperature, ° C. | 21 | 21 | 21 | 21 |
| Start of Age, Tr ° C. | 20 | | 21 | 21 |
| Heat up agitator speed, RPM | 60 | 55 | 55 | 55 |
| Max jacket temperature during | 27 | 28 | 28 | 28 |
| heat up, ° C. | | | | |
| Age temperature, ° C. | 21 | 21 | 21 | 21 |
| Age time, hr:min | 1:40 | 3:00 | 3:00 | 3:00 |
| 21 C Age agitator speed, RPM | 60 | 55 | 55 | 55 |
| Tip Speed during 21 C Age, m/s | 2.9 | 2.6 | 2.6 | 2.6 |
| total cool down time, hr:min | 4:20 | 1:50 | 1:55 | 2:10 |
| time to cool to 16° C., hr:min | 1:23 | 1:00 | 0:55 | 1:00 |
| Cool down target temperature, ° C. | 10 | 10 | 10 | 10 |
| Cool down agitator speed, RPM | 60 | 55 | 55 | 55 |
| Age temperature, ° C. | 10 | 10 | 10 | 10 |
| Age time, hr:min | 7:00 | 8:00 | 8:00 | 4:00 |
| Age agitator speed, RPM | 30 | 40 | 40 | 40 |
| Tip Speed during 10 C Age, m/s | 1.4 | 1.9 | 1.9 | 1.9 |

Filtration data:

| | | | | |
|---|---|---|---|---|
| Filter type | Nutsche | Nutsche | Nutsche | Nutsche |
| Medium # | 808 | 808 | 808 | 808 |
| Medium material | Polypropylene | Polypropylene | Polypropylene | Polypropylene |
| Medium pore size, micron | 5-10 | 5-10 | 5-10 | 5-10 |
| Slurry temperature, ° C. | 10 | 10 | 10 | 10 |
| Agitator speed, RPM | 30 | 40 | 40 | 40 |
| Main body filtration time, hr:min | 1:00 | 0:55 | 0:50 | 0:47 |
| Filtration pressure, psig | 5 | 5 | 5 | 5 |
| Water wash used, kg | 150 | 300 | 600 | 600 |
| Water wash temperature, C. | 21 | 10 | 10 | 10 |
| Wash time w/transfer, hr:min | 2:00 | | | |
| Final wash effluent conductivity, | | 12 | 9 | 4 |
| μs | | | | |
| Wet cake, kg | 7.7 | ND | ND | ND |
| Wet cake moisture content, | 52.4% | ND | ND | ND |
| calculated | | | | |
| Filtration flux, L/min/m2 | 73 | 92 | 61 | 65 |
| Blow down time, hr:min | 4 | 4 | 6 | 4.5 |
| Molar Mass by LS of wet cake | 27000 | | | |

Drying data:

| | | | | |
|---|---|---|---|---|
| Dryer type | Krauss Mafei | Nutsche | Nutsche | Nutsche |
| Jacket temperature, ° C. | 21-28.5 | RT | RT | RT |
| Vacuum | 25 mm Hg | 20 inches | Full | Full |
| 12 hr drying sample, LOD | 33.04% | ND | ND | ND |
| Drying time, hrs | 21 | 10 | 7 | 60 |
| Final KF | 6.20% | 3.71% | 2.36% | 2.36% |
| Final product temperature, ° C. | 27 | ND | ND | ND |
| Dry product, kg | 3.0 | 4.22 | 2.8 | 4.356 |
| Yield % (actual/actual) | 81.9 | 91.7 | 100.0 | 90.2 |
| Purity (HPLC Area %) | 93.9 | 93.8 | 94.1 | 93.6 |
| Molar Mass by LS of dry product | 27000 | | | |

Dissolution characteristics of re-worked material after treatment

| | | | | |
|---|---|---|---|---|
| DISSOLUTION | | | | |
| Dissolution pH | 7.63 | 6.798 | 6.794 | 6.73 |
| Solution Appearance | FT | VFT | Essentially clear | Essentially clear |
| Solution (NTU) | 23 | 2 | 2 | 23 |
| Dissolution time, hr | 1.25 | 1 | 1 | 0.75 |
| Congo Red (mAU) | 100 | 55 | 52 | 55 |

The following table summarizes additional results from the four tests summarized in the above table:

| | Re-worked Sample | | | |
|---|---|---|---|---|
| | Sample A | Sample B | Sample A′ | Sample C |
| T-1249 charged, kg | 3.7 | 4.6 | 2.8 | 4.83 |
| T-1249 Isolated, kg | 3.0 | 4.22 | 2.8 | 4.36 |
| Yield % (actual/actual) | 81.9 | 91.7 | 100.0 | 90.3 |
| Purity (HPLC Area %) | 93.9 | 93.8 | 94.1 | 93.6 |
| T-1249 NET content (%) | 89.7 | 89.4 | 89.4 | 90.8 |
| Water Content (%) | 2.3 | 2.6 | 5.44 | 2.7 |
| Molar Mass by LS of | 27000 | 9,700 | 9,300 | |
| dry product | | | | |
| Congo Red Difference | 71 | 55 | 52 | 55 |
| Absorbance 52 nm | | | | |
| Dissolution test | “Fail” | Pass | Pass | Pass |
| Compounding test | Fail | Pass | Pass | Pass |
| Reconstitution test | Fail | Pass | Pass | Pass |

### EXAMPLE 5

The following procedure describes a deaggregation process with an aging treatment that improves the filterability of a peptide sample. The process has been shown to agglomerate and “harden” the peptide particles. The precipitation was performed using 50 g of Sample A (Example 2) in a 25 L glass vessel equipped with a stirrer, jacket, and pH probe. The size of the particles was monitored using a Focused Beam Reflectance (FBRM®) instrument from Lasentec, Inc, a unit of Mettler Toledo. The FBRM® uses laser light reflectance to measure the chord length distribution (CLD) of the particles. Here is a description of the process conditions and results.

The 25 L glass vessel was charged with 11.364 L of deionized water (DIW). The temperature was adjusted to 16±1° C. 50 g of T1249 solid was charged to the vessel. The solids were dissolved by increasing the pH to 10 with 290 ml of 0.5N NaOH solution. 2500 ml Acetonitrile (ACN) was charged to a concentration of 18 v/v % ACN. The pH was further increased to 11 with 90 ml of 0.5N NaOH solution. The peptide solution was aged for 1 hour and filtered through a 0.2 μ filter membrane in 55 minutes. The total time the peptide solution was at a pH of 11 was 140 minutes. 330 ml of 5% Acetic Acid solution was charged to the precipitator at 150 RPM in 13 minutes. The final pH was 5.4.

At this point a sample of the slurry was examined on a microscope. The sample showed entities with no rigid particle structure. The precipitate at the end of precipitation appeared to be soft and gelatin-like material with no rigid particle structure under the microscope. The slurry was aged at 16±1° C. and 120 RPM 125 minutes. During this aging at 16±1° C. there was no significant change in the CLD by the FBRM®. The mean chord length remained relatively constant at 6-10 μm through out this period. The microscope picture also showed only a slight improvement in the appearance of the entities (slightly less gelatine-like appearance). After the aging at 16±1° C. the slurry was heated to 21±1° C. in 30 minutes and aged for 1 hour. The mean chord length increased by three fold to approximately 30 μm during the aging at 21±1° C. The fine particles (1 to 22 μm chord lengths) decreased significantly with a corresponding increase in large particles (22 to 100 μm chord lengths). The particles appeared to be rigid with sharp boundaries under the microscope. The particles also agglomerated during this aging period. The slurry was cooled to 10±1° C. in 1 hour and aged overnight at a reduced RPM of 90. The CLD stabilized during this aging period at 10±1° C. with no break down of the large agglomerates. The slurry was filtered on a 5 μ filter membrane using 500 mmHg vacuum in 80 minutes. In similar runs without the aging at 21±1° C. typical filtration times were several hours. Clearly the aging at 21±1° C. significantly improved the filtration characteristics of the precipitate. The wet cake was washed five times using 100 ml DIW in each wash. The conductivity of each wash was measured as 1248 μs (end of 1^{st} wash), 538 μs (end of 2^{nd} wash), 104 μs (end of 3^{rd} wash), 29 μs (end of 4^{th} wash), and 16 μs (end of 5^{th} wash). The product was dried in the filter at ambient temperature for 24 hours using 120 SCCM nitrogen sweep through the cake.

The re-worked Sample A in the 25 L precipitator showed a dissolution pH of 6.832. Upon dissolution in 1 hour, the solution was very faintly turbid. The turbidity was 4.0 NTU. During aging, particle growth occurs via acetonitrile induced particle interactions. As the solution heated up to 21° C., accelerated growth of the particles was visible. Particle growth continued through the one-hour age at 21° C. Cooling appears to stabilize the particle distribution developed at high temperature.

### EXAMPLE 6

The following procedure is used to prepare a Congo Red solution suitable for assessing, qualitatively and/or quantitatively, via colorimetric analysis the solubility and formulating characteristics of a peptide sample. A 50 mM solution of sodium phosphate buffer, pH 7.2 is prepared and filtered through a 0.22 μm Polyethersulfone membrane. 25 μM of Congo Red is dissolved in the sodium phosphate buffer with sonication. The mixture also included about 1% by volume ethanol (This can be 95% ethanol or absolute ethanol.) The solution is orange-colored.

### EXAMPLE 7

The same procedure of Example 6 is followed except that Direct Red is used instead of Congo Red.

### EXAMPLE 8

The following procedure uses the Congo Red solution of Example 6 to qualitatively calorimetrically assess the solubility, formulating, and/or degree of beta sheet aggregation characteristics of a peptide sample.

8 to 10 mg of peptide is weighed into a vial and dissolved in the orange-colored Congo Red solution to a final peptide concentration of 0.5 mg/mL (in the case of the T-1249 peptide this results in a concentration of about ˜100 μM). Sonication is used until the peptide is dissolved

The peptide/Congo Red solution is incubated at ambient temperature for 3-4 hours. If the solution is still generally orange-colored and substantially all of the peptide remains dissolved in the solution, this indicates that the peptide sample is substantially deaggregated, is soluble in aqueous solution at physiological pH and is suitable for formulating (defined above). In contrast, if the mixture turns a salmon-pink color and/or precipitated peptide is visible (a turbid supernatant indicates precipitated peptide) this indicates that the peptide sample is substantially aggregated, has poor solubility in aqueous solution at physiological pH, and will formulate poorly.

### EXAMPLE 9

The same procedure of Example 8 is followed except that Direct Red at a concentration of 25 μM or 51 μM is used instead of Congo Red.

### EXAMPLE 10

The procedure of Example 8 is useful for providing a qualitative assessment of peptide quality. However, in some instances, a more accurate assessment may be desired. For instance, in manufacturing, it may be desirable to determine whether the degree of peptide aggregation passes or fails a quantitative quality control specification with respect to aggregation/deaggregation. This kind of quantitative assessment is of particular utility for those samples in which the degree of peptide aggregation into beta sheets is moderate. In other words, a relatively low degree of beta sheet aggregation can be tolerated in a manufacturing setting, and empirical testing with respect to a particular peptide can be undertaken to set a quantitative, spectroscopic specification for samples that will be deemed to pass or fail this testing.

According to a preferred procedure, a Hewlett Packard 8453 spectrophotometer is used to obtain a blank, reference spectrum of the 50 mM solution of sodium phosphate buffer prepared in Example 6. Using the same instrument, a spectrum for the 25 μM Congo Red solution is measured over a suitable range, e.g., from about 300 nm to about 650 nm. Because the presence of peptide tends to shift the spectrum of the solution upward and to higher wavelengths to some degree, the same instrument is also used, after incubation, to scan the mixture containing the peptide over a somewhat broader, suitable range, e.g., from about 300 nm to about 700 nm. The magnitude of the difference between the spectra for the Congo Red solution and the mixture containing the peptide sample is computed at one or more appropriate wavelengths. If this difference is greater than an empirically determined quality control specification, the sample can be deemed to fail. For instance, when using a Congo Red solution to analyze a T-1249 sample, the difference is conveniently calculated at a wavelength in the range of 525 nm to 535 nm, and the quality control specification is conveniently set at a maximum difference of no more than 0.10 absorbence units in order for a sample to pass. As an alternative to computing a difference between the two spectra at this wavelength, the ratio of the magnitude of the two spectra at one or more wavelengths can be used instead. A wavelength of 505 nm is suitable for doing a ratio analysis. A greater difference (or ratio) between the two spectra indicates greater beta sheet structure content and, hence, a greater probability that the sample will not formulate properly.

The desired wavelength(s) at which to calculate spectral differences (or ratios as the case may be) will vary from dye to dye. For instance, a suitable wavelength when using Direct Red 80 is at approximately 540 nm.

### EXAMPLE 11

A number of peptide samples were subjected to staining and spectroscopic analysis in accordance with Example 10. The samples contained peptide material known to be aggregated and including a significant amount of beta sheet structure. The samples also included counterparts of these samples that had been subjected to deaggregation treatments of the present invention. The results are presented in the following table and are expressed as a difference in absorbence units between a Congo Red mixture containing the stained peptide sample and the corresponding Congo Red solution used for staining. The results show how staining with Congo Red provides an accurate qualitative and quantitative assessment of the aggregation/deaggregation characteristics of a peptide sample. Specifically, the data showed a high correlation between the magnitude of the difference between spectra and whether a peptide sample passed or failed compounding. Samples demonstrating a spectral difference of 0.1 or less at any of the wavelengths shown below, passed compounding, whereas those whose difference was greater than this tended to fail compounding.

Absorbance from Difference Spectra

| | | | | | Compounding | pH at |
|---|---|---|---|---|---|---|
| Sample | 525 nm | 528 nm | 531 nm | 535 nm | Results* | Dissolution |
| Sample A | 0.187 | 0.195 | 0.200 | 0.202 | Fail | 9.0 |
| Reworked Sample A from | 0.169 | 0.175 | 0.178 | 0.176 | Fail | 7.5 |
| Example 2 | | | | | | |
| Reworked Sample A′ from | 0.051 | 0.052 | 0.051 | 0.045 | Pass | 6.8 |
| Example 4 | | | | | | |
| Sample B | 0.261 | 0.273 | 0.282 | 0.287 | Fail | 9.4 |
| Reworked Sample B from | 0.041 | 0.038 | 0.034 | 0.025 | Pass | 6.8 |
| Example 3 | | | | | | |
| Sample C | 0.128 | 0.126 | 0.122 | 0.111 | Fail | 9.2 |
| Reworked Sample C from | 0.054 | 0.055 | 0.053 | 0.047 | Pass | 6.8 |
| Example 4 | | | | | | |

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1. A method of processing a peptide, comprising the steps of:
(a) combining a portion of the peptide with a calorimetric reagent comprising a dye that calorimetrically and selectively interacts more strongly with a first structure of the peptide relative to a second structure of the peptide, wherein the first structure is substantially insoluble in aqueous media at physiological pH, and wherein the second structure is substantially soluble in aqueous media at physiological pH;
(b) obtaining calorimetric information from step (a) that is indicative of whether the peptide sample contains an undesired amount of the first structure;
(c) using the information of step (b) to determine whether to subject the peptide to a deaggregation treatment.

2. The method of claim 1 wherein the calorimetric reagent comprises an azo dye.

3. The method of claim 1 wherein the colorimetric reagent comprises a diazo dye.

4. The method of claim 2 wherein the azo dye comprises a napthalenesulfonic acid group.

5. The method of claim 2 wherein the azo dye is selected from Congo Red and Direct Red.

6. The method of claim 2 wherein the azo dye comprises Congo Red.

7. The method of claim 2 wherein the azo dye comprises Direct Red.

8. The method of claim 1, wherein the peptide is selected from one or more of T-1249 peptide, a T-1249 fragment, and a counterpart thereof.

9. The method of claim 1, wherein the peptide has HIV fusion inhibitor activity.

10. The method of claim 1, wherein the peptide comprises beta sheet structure.

11. The method of claim 1 wherein step (b) comprises obtaining spectroscopic information.

12. The method of claim 1 wherein the colorimetric reagent has a pH in the range of from about 6 to about 8.

13. The method of claim 1 wherein step (c) comprises comparing the calorimetric information of the sample to calorimetric information of a reference sample.

14. The method of claim 13, wherein comparing comprises determining a quantitative difference between a spectral peak of the sample and a spectral peak of the reference sample at at least one wavelength.


SEQUENCE LISTING (35 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 8 aa, Artificial
  Chemically synthesized peptide fragment
YTSLIHSL

SEQ ID NO: 3 - protein, 15 aa, Artificial
  Chemically synthesized peptide fragment
YTSLIHSLIEESQNQ

SEQ ID NO: 4 - protein, 16 aa, Artificial
  Chemically synthesized peptide fragment
YTSLIHSLIEESQNQQ

SEQ ID NO: 5 - protein, 18 aa, Artificial
  Chemically synthesized peptide fragment
YTSLIHSLIEESQNQQEK

SEQ ID NO: 6 - protein, 7 aa, Artificial
  Chemically synthesized peptide fragment
IEESQNQ

SEQ ID NO: 7 - protein, 8 aa, Artificial
  Chemically synthesized peptide fragment
IEESQNQQ

SEQ ID NO: 8 - protein, 20 aa, Artificial
  Chemically synthesized peptide fragment
QEKNEQELLELDKWASLWNW

SEQ ID NO: 9 - protein, 21 aa, Artificial
  Chemically synthesized peptide fragment
QEKNEQELLELDKWASLWNWF

SEQ ID NO: 10 - protein, 7 aa, Artificial
  Chemically synthesized peptide fragment
EKNEQEL

SEQ ID NO: 11 - protein, 10 aa, Artificial
  Chemically synthesized peptide fragment
EKNEQELLEL

SEQ ID NO: 12 - protein, 20 aa, Artificial
  Chemically synthesized peptide fragment
EKNEQELLELDKWASLWNWF

SEQ ID NO: 13 - protein, 17 aa, Artificial
  Chemically synthesized peptide fragment
NEQELLELDKWASLWNW

SEQ ID NO: 14 - protein, 18 aa, Artificial
  Chemically synthesized peptide fragment
NEQELLELDKWASLWNWF

SEQ ID NO: 15 - protein, 12 aa, Artificial
  Chemically synthesized peptide fragment
LELDKWASLWNW

SEQ ID NO: 16 - protein, 13 aa, Artificial
  Chemically synthesized peptide fragment
LELDKWASLWNWF

SEQ ID NO: 17 - protein, 9 aa, Artificial
  Chemically synthesized peptide fragment
DKWASLWNW

SEQ ID NO: 18 - protein, 10 aa, Artificial
  Chemically synthesized peptide fragment
DKWASLWNWF

SEQ ID NO: 19 - protein, 19 aa, Artificial
  Chemically synthesized peptide fragment
EKNEQELLELDKWASLWNW

SEQ ID NO: 20 - protein, 39 aa, Artificial
  Chemically synthesized peptide fragment
WQEWEQKITALLEQAQIQQEKNEYELQKLDKWASLWEWF

SEQ ID NO: 21 - protein, 20 aa, Artificial
  Chemically synthesized peptide fragment
WQEWEQKITALLEQAQIQQE

SEQ ID NO: 22 - protein, 18 aa, Artificial
  Chemically synthesized peptide fragment
KNEYELQKLDKWASLWEW

SEQ ID NO: 23 - protein, 19 aa, Artificial
  Chemically synthesized peptide fragment
KNEYELQKLDKWASLWEWF

SEQ ID NO: 24 - protein, 19 aa, Artificial
  Chemically synthesized peptide fragment
IQQEKNEYELQKLDKWASL

SEQ ID NO: 25 - protein, 17 aa, Artificial
  Chemically synthesized peptide fragment
WQEWEQKITALLEQAQI

SEQ ID NO: 26 - protein, 16 aa, Artificial
  Chemically synthesized peptide fragment
WQEWEQKITALLEQAQ

SEQ ID NO: 27 - protein, 19 aa, Artificial
  Chemically synthesized peptide fragment
QQEKNEYELQKLDKWASLW

SEQ ID NO: 28 - protein, 18 aa, Artificial
  Chemically synthesized peptide fragment
QQEKNEYELQKLDKWASL

SEQ ID NO: 29 - protein, 20 aa, Artificial
  Chemically synthesized peptide fragment
IQQEKNEYELQKLDKWASLW

SEQ ID NO: 30 - protein, 12 aa, Artificial
  Chemically synthesized peptide fragment
WQEWEQKITALL

SEQ ID NO: 31 - protein, 14 aa, Artificial
  Chemically synthesized peptide fragment
EQAQIQQEKNEYEL

SEQ ID NO: 32 - protein, 12 aa, Artificial
  Chemically synthesized peptide fragment
QKLDKWASLWEW

SEQ ID NO: 33 - protein, 13 aa, Artificial
  Chemically synthesized peptide fragment
QKLDKWASLWEWF

SEQ ID NO: 34 - protein, 27 aa, Artificial
  Chemically synthesized peptide fragment
EQAQIQQEKNEYELQKLDKWASLWEWF

SEQ ID NO: 35 - protein, 31 aa, Homo sapiens
HAEGTFTSDVSSYLEGQAAKEFIAWLVKGRG